(19) United States
(12) Patent Application Publication
(10) Pub. No.: US 2025/0194958 A1
(43) Pub. Date: Jun. 19, 2025

(54) WEARABLE DEVICES AND SYSTEMS TO MONITOR VITAL SIGNS USING ACCELEROMETER SIGNALS

(71) Applicant: Analog Devices International Unlimited Company, Limerick (IE)

(72) Inventors: Kate Danielle FISCHL, Jamaica Plain, MA (US); Christoph KAEMMERER, Munchen (DE); Michael R. PRICE, Medford, MA (US); Xiao Yu WANG, Watertown, MA (US); Lauren Emily MENTZER, Boston, MA (US); Sunrita PODDAR, Jamaica Plain, MA (US)

(21) Appl. No.: 19/064,616

(22) Filed: Feb. 26, 2025

Related U.S. Application Data
(63) Continuation of application No. PCT/EP2023/073572, filed on Aug. 28, 2023.
(60) Provisional application No. 63/401,206, filed on Aug. 26, 2022.

(30) Foreign Application Priority Data
Jul. 6, 2024 (IN) 202441051837

(51) Int. Cl. A61B 5/11; A61B 5/00

(52) U.S. Cl. CPC A61B 5/1126; A61B 5/6823; A61B 5/7225; A61B 5/742; A61B 2562/0219

(57) ABSTRACT

Technologies are provided for vital sign monitoring using accelerometer signals. The accelerometer signals may be obtained from a wearable device in contact with a subject being monitored. In some aspects, magnitude of the acceleration signals permits obtaining robust determination of a vital sign, such as heart rate or respiration rate, of the subject.

### CROSS-REFERENCE TO RELATED APPLICATIONS

This application is a by-pass continuation of International Patent Application No. PCT/EP2023/073572, filed Aug. 28, 2023, which application claims the benefit of and priority to U.S. Provisional Patent Application No. 63/401,206, filed Aug. 26, 2022, and this application also claims the benefit of and priority to India Provisional Patent Application No. 20/244,1051837, filed Jul. 6, 2024, the contents of which applications are hereby incorporated herein by reference in their entireties.

### BACKGROUND

Humans and other mammals exhibit three major vital signs—temperature, respiration, and pulse—that can readily convey whether a subject is alive. These and other vital signs are associated with vital bodily functions, and can be used to assess a state of health of a subject beyond a straightforward determination that the subject is alive.

Monitoring of vital signs of a subject is thus a ubiquitous tool in healthcare, and is commonly accomplished by tethering the subject to monitoring equipment. As such, commonplace monitoring of vital signs can be complicated and rather invasive, especially in situations where the subject is an infant, a young child, or an elder. Even if tethering monitoring equipment to a subject could be readily accomplished, there are situations where the monitoring of vital signs is necessary over extended periods of times, as it would be the case for subjects undergoing non-ambulatory care at a hospital or another type of medical facility. In those situations, monitoring the vital signs of a subject may be disrupted due to movement of the subject or other factors, such as discomfort or pain, that result in the subject becoming untethered to the monitoring equipment. Not only can disruptions yield unreliable data, but in a subject under intensive care, for example, disruptions in the monitoring of vital signs can be detrimental, if not harmful, to the subject.

Therefore, several technical challenges remain in the field of monitoring of vital signs and the technologies for such monitoring. Improved technologies that address those challenges are desirable.

### BRIEF DESCRIPTION OF THE DRAWINGS

The accompanying drawings form part of the disclosure and are incorporated into the subject specification. The drawings illustrate example aspects of the disclosure and, in conjunction with the following detailed description, serve to explain at least in part various principles, features, or aspects of the disclosure. Some aspects of the disclosure are described more fully below with reference to the accompanying drawings. However, various aspects of the disclosure can be implemented in many different forms and should not be construed as limited to the implementations set forth herein. Like numbers refer to like elements throughout.

FIG. 1 is a block diagram of an example of a system for contactless monitoring of vital signs, in accordance with one or more aspects of this disclosure.

FIG. 2A presents plots of examples of observed accelerometer signals for three axes of an accelerometer device that can be part of the system shown in FIG. 1, in accordance with one or more aspects of this disclosure.

FIG. 2B presents other plots of examples of observed accelerometer signals for three axes of an accelerometer device that can be part of the system shown in FIG. 1, in accordance with one or more aspects of this disclosure.

FIG. 2C presents an example of raw accelerometer signals and detrended

accelerometer signals for a particular channel of an accelerometer device that can be part of the system shown in FIG. 1, in accordance with one or more aspects of this disclosure.

FIG. 2D presents yet other plots of examples of observed accelerometer signals for three axes of an accelerometer device that can be part of the system shown in FIG. 1, an example of observed ECG signal, and the magnitude of accelerometer signal determined using the observed accelerometer signals, in accordance with one or more aspects of this disclosure.

FIG. 2E illustrates an example adaptive window approach used to determine peaks and troughs in a conditioned acceleration magnitude signal, in accordance with aspects of this disclosure.

FIG. 3 is an example of a user interface (UI) where heart rate and respiration rate are presented as a function of time, in accordance with one or more aspects of this disclosure. The UI can be presented during the contactless monitoring of vital signs described herein.

FIG. 4 is a block diagram of a contactless monitoring unit that contains an example of a system for contactless monitoring of vital signs, in accordance with one or more aspects of this disclosure.

FIG. 5 a diagram of an example of a system for contactless monitoring of vital signs, in accordance with one or more aspects of this disclosure.

FIG. 6A is a diagram of an example system for contact monitoring of vital signs in accordance with one or more aspects of this disclosure.

FIG. 6B is a diagram of another example system for contact monitoring of vital signs in accordance with one or more aspects of this disclosure.

FIG. 6C is a diagram of yet another example system for contact monitoring of vital signs in accordance with one or more aspects of this disclosure.

FIG. 7 is a block diagram of an example of a computing device that can provide various functionality of contactless monitoring of vital signs in accordance with one or more aspects of this disclosure.

FIG. 8 is a schematic block diagram of a processing platform to monitor vital signs using accelerometer signals, in accordance with one or more aspects of this disclosure.

FIG. 9A is a block diagram of an example process flow for detection of a subject in bed using acceleration signals, in accordance with one or more aspects of this disclosure.

FIG. 9B is a block diagram is a block diagram of an example of a system for contactless monitoring of vital signs, in accordance with one or more aspects of this disclosure.

FIG. 10 depicts blocks representing groups of acceleration samples in buffer, in accordance with one or more aspects described herein.

FIG. 11 is a schematic block diagram of a predictive model for detection of a subject in bed, in accordance with one or more aspects of this disclosure.

FIG. 12 is a schematic block diagram of another example of a predictive model for detection of a subject in bed, in accordance with one or more aspects of this disclosure.

FIG. 13 illustrates a prediction map generated with an example predictive model for detection of subject in bed, in accordance with one or more aspects of this disclosure.

FIG. 14 illustrates how the waveform features can be useful for detection of a subject in accordance with aspects of this disclosure.

### DETAILED DESCRIPTION

Embodiments of the disclosure address, among other technical challenges, the issue of monitoring vital signs of a subject. Commonplace monitoring of vital signs involves tethering a subject to dedicated monitoring equipment. For that reason, commonplace monitoring of vital signs can be complicated and rather invasive, especially in situations where the subject being monitored is an infant, a young child, or an elder. Even if tethering monitoring equipment to the subject could be readily accomplished, there are situations where the monitoring of vital signs is necessary over extended periods of times, without interruption. In those situations, monitoring the vital signs of the subject may be disrupted due to movement of the subject or other factors, such as discomfort or pain, that result in the subject becoming untethered to the monitoring equipment. Besides yielding unreliable data, in some circumstances, disruptions in the monitoring of vital signs can be detrimental, if not altogether harmful, to the subject.

Further, vital signs such as heart rate (HR) and respiration rate (RR) are useful metrics to obtain a portrait of a person's health. Vital signs estimates are often obtained manually and non-continuously, such as in hospitals (outside the ICU), where measurements are taken periodically by an aide, a nurse, or a clinician. Undetected deterioration of vital signs can be detrimental, if not altogether harmful or even fatal.

The technologies described herein provide systems, devices, computer-implemented methods, and computer program products that, individually or in combination, permit contactless monitoring of vital signs of a subject. As is used herein, the term “contactless monitoring” refers to monitoring that avoids tethering the subject in any way to monitoring equipment.

In accordance with aspects of the present disclosure vital signs can be monitored continuously or nearly continuously using the accelerometer device coupled with a computing device. The monitoring can be implemented in accordance with various scenarios, including (A) Contactless (or non-contact), in-bed scenario where the accelerometer device is placed in a bed or is otherwise attached to the bed, and vital signs of a subject are monitored when the subject lies on the bed. (B) Contact, on-wearable-device, where the accelerometer device is integrated into a wearable device (e.g., a patch, chest-strap device, or the like), and vital signs may be estimated (continuously or otherwise) as the subject wears the wearable device. In some cases, the wearable device implements the approaches described herein. In other cases, the wearable device is functionally coupled with one or more computing devices that can implement the approaches described herein. The computing device(s) are remotely located relative to the wearable device.

Other aspects of the disclosure provide one or more approaches for determining that a subject is present in or absent from a bed. The approach(es) also can user the accelerometer device and a data indicative of magnitude of acceleration signals in respective channels of the accelerometer device.

As is described in greater detail below, embodiments of the technologies described herein can monitor vital signs in contactless fashion by using acceleration signals corresponding to respective accelerometer channels of an accelerometer device. The accelerometer device is mechanically coupled with a subject via a solid medium, where the subject is being monitored in contactless fashion in accordance with aspects described herein. The solid medium couples the accelerometer device and the subject by propagating movement of the subject towards the accelerometer device. In some cases, the accelerometer device is integrated into the solid medium, which medium is configured to support the subject in laydown position and/or an upright sitting position, for example. In addition, or in some cases, the solid medium deforms under load from a subject-that is, the solid medium can be deformed essentially elastically by a weight of the subject. By being deformable, the solid medium permits propagating motion from the subject to the accelerometer device. Thus, the solid medium mechanically couples the subject and accelerometer device with one another. In some cases, depending on the vital sign being monitored (e.g., heart rate), the solid medium can be rigid and can support the accelerometer device instead of the subject. For example, the solid medium can be a frame of furniture configured to receive, and hold, another solid medium that deforms under load by the subject. The accelerometer device is attached to such a frame.

The accelerometer device also is functionally coupled to a computing device having one or more processors and other computing resources. A communication architecture permits the transport of accelerometer data, including the acceleration signals, from the accelerometer device to the computing device. In some cases, the accelerometer device is deployed separately from the computing device. In other cases, the accelerometer device and the computing device are integrated into a single module. The single module may be a wearable device. An example of the wearable device includes a patch that can be adhered to a subject.

The computing device can operate on the acceleration signals in order to generate conditioned accelerometer signals. The conditioned accelerometer signals can be physiological signals corresponding to vital bodily functions associated with vital signs being monitored in the contactless fashion as is described herein. Without intending to be bound by theory or modeling, the conditioned accelerometer signals represent such physiological signals because the vital bodily functions being monitored involve motion of the body of the subject, and the motion can be monitored in contactless fashion using the accelerometer device coupled to the computing device. Such a motion does not result in substantive displacement of the subject on a surface of the solid medium. That is, the vital bodily functions involve movement of body parts (e.g., chest wall) and/or body portions (e.g., torso) that essentially maintain the subject at rest on the surface of the solid medium.

The physiological signals correspond to both respiration and cardiac function, which are vital bodily functions that exhibit cyclical characteristics.

Hence, the computing device can generate a dataset indicative of peaks present in the magnitude of accelerometer signals, where the accelerometer signals correspond to respective accelerometer channels. The computing device can determine, using the dataset, first estimates of a vital sign of a subject. The vital sign can be respiration rate or heart rate, for example.

The computing device can then provide the observed estimate of the vital sign. In some cases, the computing device can provide the observed estimate of the vital sign by causing a display device to present the observed estimate of the vital sign in a user interface accessible to the subject and/or a healthcare provider of the subject.

Because the accelerometer device that is mechanically coupled to the subject can generate acceleration signals over time, at a defined sampling rate, the computing device can determine additional observed estimates of the vital sign over time, as the acceleration signals become available to the computing device. Thus, in sharp contrast to some existing, non-contact technologies that monitor vital signs, the technologies described herein provide estimates of a vital sign contemporaneously with the vital bodily function associated with the vital sign. Accordingly, the vital sign is monitored in contactless fashion and in real-time or nearly real-time, without resorting to averaging estimates of the vital sign of arbitrarily defined time periods. As a result, the contactless monitoring of vital signs that is described in this disclosure drastically improves the technological field of vital sign monitoring. Indeed, the technologies described herein avoid the use of dedicated and rather costly equipment, and also maintain a timely assessment of the vital signs of the subject being monitored.

Various aspects in accordance with this disclosure are described in connection with a bed, such as residential bed or a hospital bed, as furniture where the contactless monitoring of vital signs is implemented. The disclosure is not limited in that respect. Indeed, the principles and practical applications of the contactless monitoring of vital signals in accordance with aspects of this disclosure can be implemented in other types of furniture, include recliner chairs, sofas, cribs, cots, mats, blankets, cloaks, and the like. Indeed, the contactless monitoring of vital signs that is described herein can be implemented in any solid medium that is configured to support a subject in laydown position, and/or an upright position, or is configured to be disposed so as to contact a torso of the subject by supporting the subject or covering the subject. In this disclosure, a “subject” is a human being or another type of animal. Non-human animals can include dogs, cats, horses, chimpanzees and other non-human primates, and livestock (such as cows, pigs, sheep, and the like).

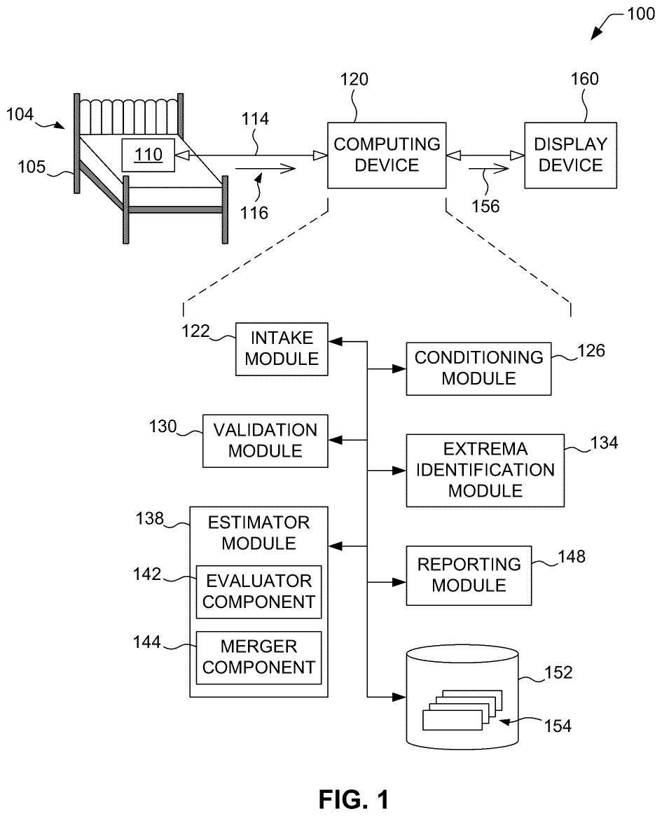

FIG. 1 is a block diagram of an example of a system 100 for non-contact monitoring of vital signs of a subject, in accordance with one or more aspects of this disclosure. The example system 100 includes a bed 104 where a subject (not depicted) can lie down or sit upright to be monitored. It is noted that in situations where the subject is sitting upright on the bed 104, a particular type of vital sign may not be monitored as long as the subject remains in such a position. The cyclic motion involved in breathing and circulation of blood within the body of the subject causes a time-dependent deformation of a solid medium (e.g., a mattress or mattress topper) that constitutes the bed 104. By monitoring the time-dependent deformation of the solid medium, RR and HR of the subject can be monitored in contactless fashion. Here, consistent with the foregoing characterization of “contactless monitoring,” monitoring implemented in “contactless fashion” refers to monitoring that avoids tethering the subject in any way to monitoring equipment.

To monitor RR and HR of the subject, the bed 104 is fitted with an accelerometer device 110 that is mechanically coupled with the subject via the solid medium that constitutes, at least partially, the bed 104. Without intending to be bound by theory, the solid medium propagates movement of the subject to the accelerometer device 110. The accelerometer device 110 can be assembled in numerous ways within or about the bed 104. For example, the accelerometer device 110 can be embedded within the solid medium (e.g., a mattress). As another example, in case the bed 104 includes a mattress topper, the accelerometer device 110 can be disposed on a surface at the interface between a mattress of the bed 104 and the mattress topper. In yet another example, the accelerometer device 110 can be embedded within a pillow that rests on a mattress of the bed 104. In still another example, the accelerometer device 110 can be affixed to the solid medium (e.g., a bed frame 105 of the bed 104).

The accelerometer device 110 is a low-noise accelerometer device that is configured to generate measurement signals representative of movement of the body of the subject as the subject breathes and has an active cardiovascular system while lying or sitting upright on the bed 104. The measurement signals can be referred to as accelerometer signals and form a time series of acceleration values. Each acceleration value can be referred to as a sample. The accelerometer device 110 generates the acceleration values at a defined sampling rate (or data output rate). Examples of the sampling rate are 250 Hz and 256 Hz.

The accelerometer device 110 is a low-noise accelerometer device and can be a three-axis accelerometer device, such as a three-axis microelectromechanical systems (MEMS) accelerometer. At a time of sampling acceleration, the three-axis accelerometer device generates three acceleration signals corresponding to three orthogonal axes, such as the axes x, y, and z of a Cartesian coordinate system. Each of those axes is referred to as a channel. Thus, over a time period, the accelerometer device 110 is configured to generate first accelerometer signals corresponding to a first channel α (x, y, or z), second accelerometer signals corresponding to a second channel β, and third accelerometer signals corresponding to a third channel γ (x, y, or z), where α≠β≠γ. The inertial measurements are collectively representative of (i) an orientation of the three-axis accelerometer device relative to gravity when the accelerometer device is at rest and (ii) a rectilinear acceleration vector. When the accelerometer device 110 moves in response to movement of the subject's body, the accelerometer device 110 can measure the generated rectilinear acceleration vector. Without intending to be bound by modeling, during contactless monitoring of vital signs of a subject in accordance with this disclosure, the rectilinear acceleration vector has negligible magnitude.

The accelerometer device 110 is functionally coupled to a computing device 120. The computing device 120 can include computing resources (not shown) including one or more processing units, each including at least one processor; one or more memory devices, one or more interfaces that can permit the exchange of data and signaling between a processing unit and a memory device and/or between the computing device and an external device; a power supply; a combination of the foregoing; and/or similar resources. Although the computing device is illustrated as being external to the bed 104, the disclosure is not limited in that respect and other configurations are contemplated.

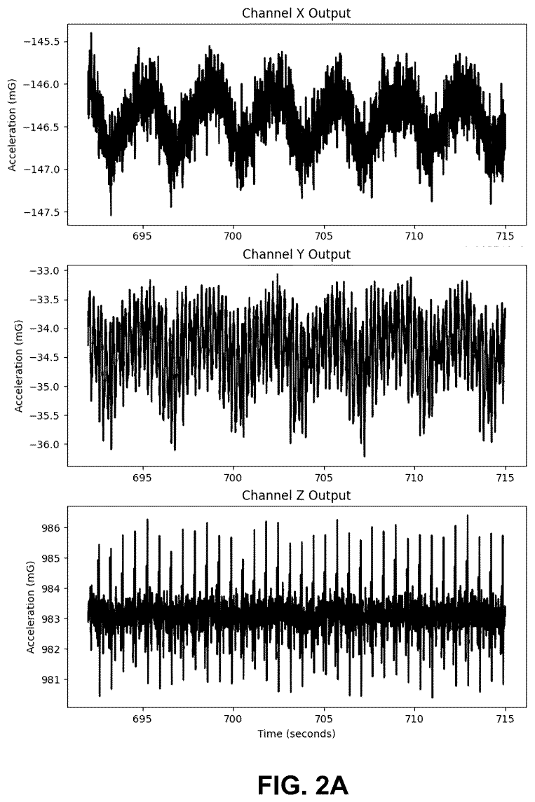

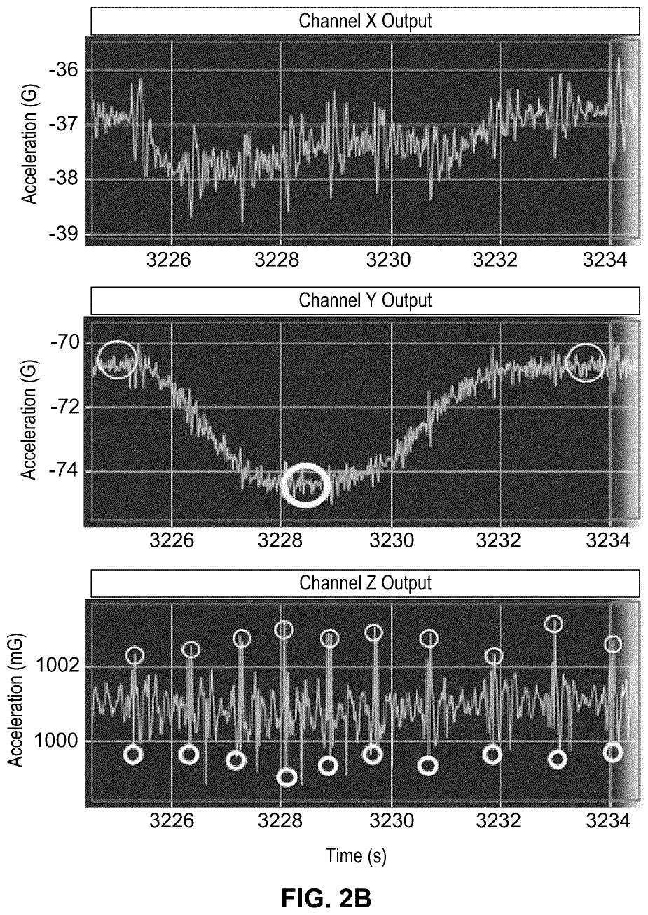

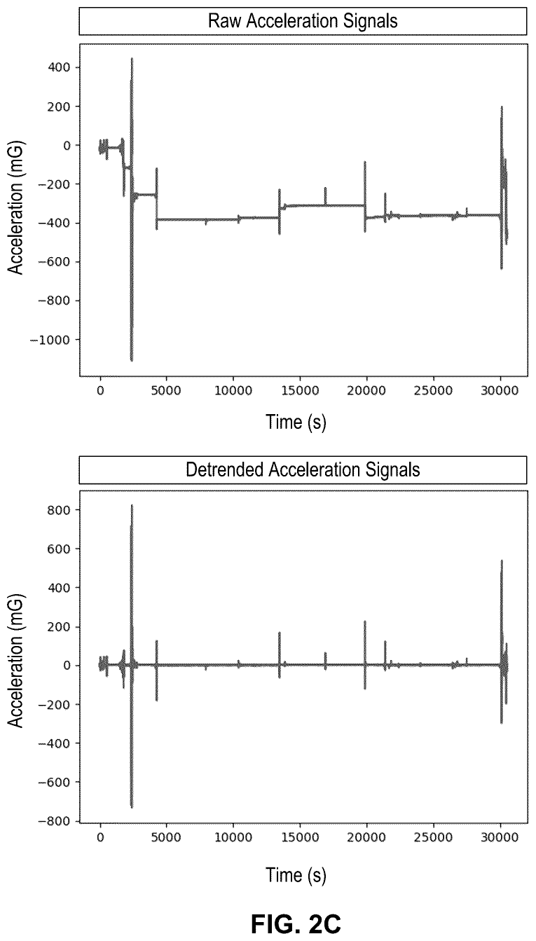

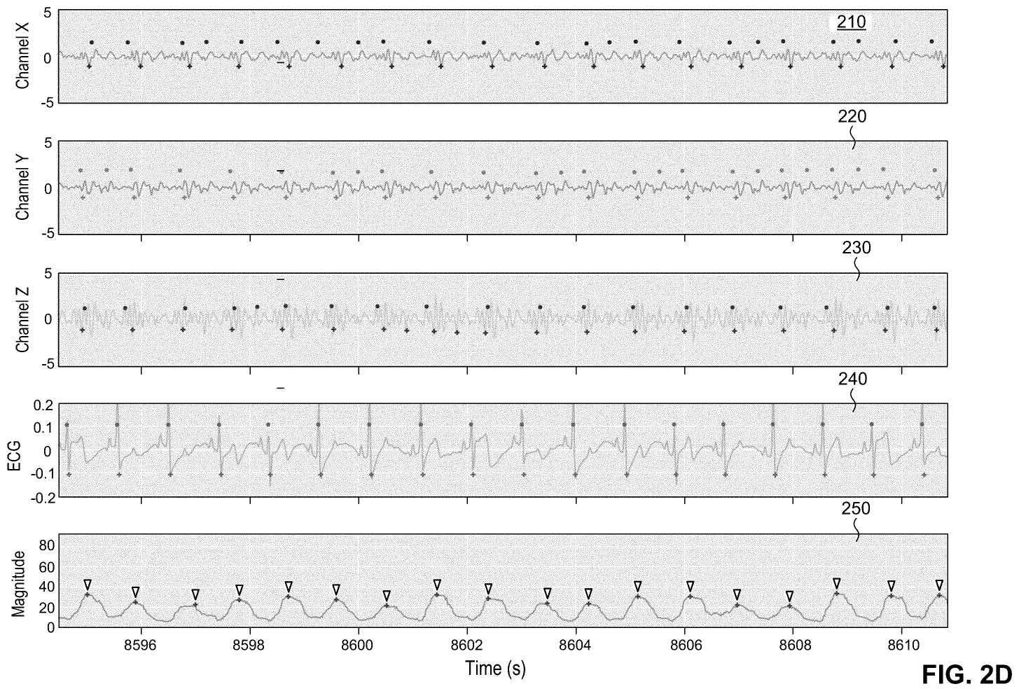

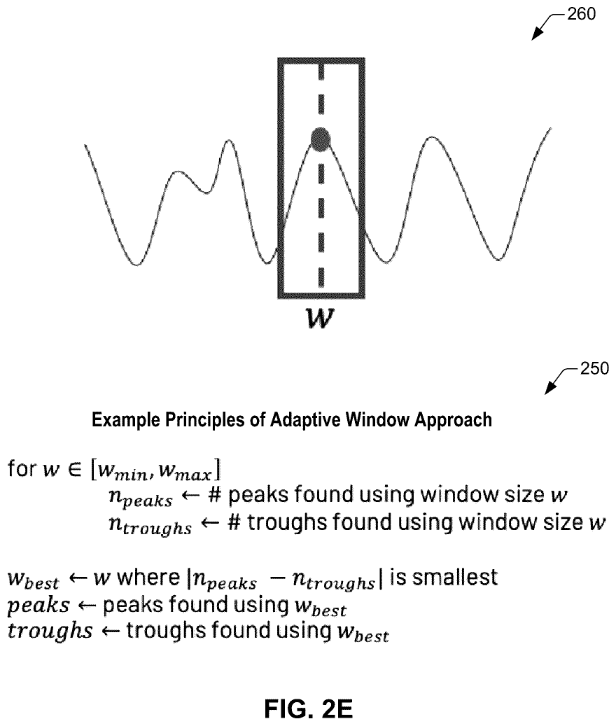

A communication architecture 114 permits the functional coupling between the accelerometer device 110 and the computing device 120. The particular structure of the communication architecture 114 depends on the type of computing device 120 that is coupled to the accelerometer device 110. Regardless of its particular structure, the communication architecture 114 includes an uplink that can transport accelerator signals 116 from the accelerometer device 110 to the computing device 120, as the accelerator signals 116 are generated. Hence, over time, the accelerometer device 110 can send a first time series {aₓ} of acceleration values corresponding to channel x, a second time series {a_{y}} of acceleration values corresponding to channel y, and a third time series {a_{z}} of acceleration values corresponding to channel z. In one configuration, channel x and channel y correspond to in-plane acceleration on a plane perpendicular to gravity, and channel z correspond to acceleration on an axis parallel to gravity. The acceleration values in each of the first, second, and third time series are separated in time domain by a time interval equal to the reciprocal of the defined sampling rate of the accelerometer device 110. Thus, at a measurement time tₙ, the accelerometer device 110 can send a first signal indicative of an acceleration value aₓ[n] for channel x, a second signal indicative of an acceleration value a_{y}[n], and a third signal indicative of an acceleration value a_{z}[n]. Simply as an illustration, FIG. 2A presents example plots of observed {aₓ}, {a_{y}}, and {a_{z}} during an example time interval with a subject lying on the bed 104. The example time interval is merely illustrative. FIG. 2B presents other example plots of observed {aₓ}, {a_{y}}, and {a_{z}} during another example time interval with a subject lying on the bed 104. The other example time interval is merely illustrative. Simply for the sake of representation, in the example plot for {a_{y}} (the plot labeled as “Channel Y Output”) open circles with a thin boundary mark respective peaks in RR. Further, in the same plot, an open circle with a thick boundary line marks a trough in RR.

With further reference to FIG. 1, the computing device 120 can obtain accelerometer signals from the accelerometer device 110 via the communication architecture 114. To the end, as is illustrated in FIG. 1, the computing device 120 can include an intake module 122 that can receive the accelerometer signals.

A subject being monitored can, over time, change positions in the bed 104. Thus, a baseline of accelerometer signals corresponding to a channel can change over time. In addition, a bodily function of the subject other than the bodily function being monitored for vital signs can introduce noise in the accelerometer signals being used to monitor the vital signs. For example, cardiac function may introduce noise that can distort the monitoring of pulmonary ventilation. In addition, or in some cases, equipment (a refrigerator, for example) operating in proximity of the bed 104 and coupled (mechanically, electrically, and/or in other way) to the bed 104 also can introduce noise that can distort the monitoring of a particular vital sign. Accordingly, the computing device 120 can condition the accelerometer signals received by the computing device 120 before determining an estimate of a vital sign of the subject in the bed 104. Because in embodiments of this disclosure the monitoring of vital signs of the subject is contemporaneous with the generation of the accelerometer signals, the computing device 120 can condition the accelerometer signals as the accelerometer signals are received at the computing device 120. Conditioning of the accelerometer signals can result in the removal of components of accelerometer signal that are not germane or otherwise representative of the vital sign being monitored. Accordingly, an accelerometer signal that has been conditioned in accordance with aspects described herein can be a physiological signal corresponding to vital bodily functions associated with vital signs being monitored in contactless fashion.

To condition an accelerometer signal, and thus generate a physiological signal, the computing device 120 can include a conditioning module 126. The conditioning module 126 can operate on the accelerometer signals to detrend the accelerometer signals, resulting in detrended accelerometer signals. Detrending an accelerometer signal includes subtracting a moving average of the accelerometer signal. The moving average can change over time due to changes in position of the monitored subject in the bed 104. Hence, the detrended accelerometer signals lack baseline drift. Detrending the accelerometer signals ultimately removes the acceleration due to gravity from the accelerometer signals. Simply as an illustration, FIG. 2C presents accelerometer signals as received (referred to as raw accelerometer signals) and detrended accelerometer signals for a particular channel of the accelerometer device 110, during an example time interval. The example time interval spans approximately 8.5 hours, which can correspond to a desirable amount of asleep time in a night.

As part of conditioning accelerometer signals, and thus generate physiological signals, the conditioning module 126 (FIG. 1) also can operate on detrended accelerometer signals to filter the detrended accelerometer signals, resulting in filtered accelerometer signals. In order to yield filtered accelerometer signals that are appropriate for determining estimates of respiration rate, the conditioning module 126 can filter the detrended accelerometer signals by applying a low-pass filter configured to remove frequencies exceeding a cutoff frequency. The cutoff frequency is configured to a value that ensures that all likely frequencies of pulmonary ventilation in an adult healthy human are contained in the filtered accelerometer signal. Those likely frequencies can be within a breathing bandwidth that can range from about 0.08 Hz to about 0.42 Hz, for example. By filtering the detrended acceleration signals in such a fashion, frequency components associated with other bodily functions besides the particular bodily function being monitored (e.g., breathing, or cardiac function) can be removed from the detrended acceleration signals. In other words, filtering the detrended acceleration signals can retain respiratory information in the fundamental frequency, and eliminates higher frequencies associated with other vital bodily functions. In one example configuration, the cutoff frequency is 1.0 Hz and the low-pass filter is a Butterworth filter having four taps. In another example configuration, the low-pass filter is a bidirectional infinite impulse response (IIR) filter with a corner frequency of 0.2 Hz. The disclosure is, of course, not limited to such example configurations.

Similarly, to yield filtered accelerometer signals that are appropriate for determining estimates of heart rate, the conditioning module 126 can filter detrended accelerometer signals by applying a band-pass filter configured to remove frequencies exceeding a cutoff frequency within a pulse bandwidth and frequencies less than a lower bound of the pulse bandwidth. In some configurations, the band-pass filter is a Butterworth filter that removes frequencies outside the interval from 0.5 Hz to 20 Hz, to remove effects of the respiratory modulation and other higher frequency effects.

The sampling rate of the accelerometer device 110 is greater than the RR and/or the HR of the subject being monitored. Thus, in some cases, the conditioning module 126 can optionally decimate detrended accelerometer signals before filtering those signals as is described above. In one example, by decimating a detrended accelerometer signal, the conditioning module 126 can downsample the detrended acceleration signal to 5 Hz to monitor respiration rate. In another example, by decimating a detrended accelerometer signal, the conditioning module 126 can downsample the detrended acceleration signal to 50 Hz to monitor heart rate. For heart rate, 50 Hz is used instead of 5 Hz because HR has a shorter period than RR.

Conditioning the accelerometer signals received at the computing device 120 results in conditioned accelerometer signals for respective accelerometer channels. Namely, in cases where the accelerometer device 110 is a low-noise three-axis accelerometer device, the conditioning module 126 can generate a first conditioned accelerometer signal corresponding to channel x, a second conditioned accelerometer signal corresponding to channel y, and third accelerometer signals corresponding to channel z. As mentioned, the conditioned accelerometer signals can be physiological signals corresponding to vital bodily functions associated with vital signs being monitored in the contactless fashion as is described herein.

Because the monitoring of vital signs in accordance with this disclosure is contemporaneous with the generation of the accelerometer signals while a subject sleeps or is lying in the bed 104 at rest, accelerometer signals received at the computing device 120 may, in some instances, have poor quality due to noise or other factors, such as the subject being absent from the bed 104. As such, the computing device 120 can determine if a received accelerometer signal, e.g., a current sample a_{μ}[n](μ=x, y, or z), is valid or otherwise suitable for inclusion in the analysis involved in the determination of a vital sign. To that end, in some cases, as is illustrated in FIG. 1, the computing device 120 can include a validation module 130 that can determine if an energy of the received accelerometer signal has a magnitude within a defined range. A valid accelerometer signal has an energy within the defined range. Conversely, an invalid accelerometer signal has an energy outside the defined range. Without intending to be bound by theory, the energy E[n] of a current acceleration signal, a_{μ}[n], is defined in terms of an energy contribution of the immediately prior acceleration signal, a_{μ}[n−1], and another energy contribution of a_{μ}[n]. Defining E[n] in such a fashion provides added computation stability to the techniques described herein. The lower bound (or threshold) of the defined range serve as a check on whether a subject is present in the bed 104. The upper bound serves as a guard on movement that causes large spikes in the accelerometer data. Accordingly, the upper bound can be several orders of magnitude greater than the lower bound.

The validation module 130 can determine, using conditioned accelerometer signals, the presence or absence of valid signals in respective accelerometer channels and can update valid signal datasets (e.g., time series of valid signals) for the respective accelerometer channels. Each valid signal dataset of the multiple valid datasets defines a conditioned acceleration waveform in a particular channel of the respective channels. The conditioned acceleration waveform is a physiological signal corresponding to vital bodily functions associated with a vital sign (e.g., RR or HR) being monitored in contactless fashion. That is, in case the accelerometer device 110 is a three-axis accelerometer device, a first valid dataset of the multiple datasets defines a first conditioned acceleration waveform (or first physiological signals) for channel x, a second valid dataset of the multiple datasets defines a second conditioned acceleration waveform (or second physiological signals) for channel y, and a third valid dataset of the multiple datasets defines a third acceleration waveform (or third physiological signals) for channel z.

A conditioned acceleration waveform defined by a valid signal dataset for an accelerometer channel (x, y, or z, for example) can be substantially cyclic in time because pulmonary ventilation and blood circulation in a subject are substantially cyclic. Such cyclic characteristic is visible in signals shown in FIG. 2D. Plot 210, plot 220, and plot 230 present, respectively, example observed {aₓ}, {a_{y}}, and {a_{z}} during an example time interval with a subject lying on the bed 104. In each one of plot 210, plot 220, and plot 230, peaks and troughs are marked with solid circles and crosses, respectively. Similar to other diagrams, the example time interval shown in FIG. 2D is merely illustrative.

Conditioned acceleration waveforms for channels x, y, and z can be used to generate a conditioned acceleration magnitude waveform that is a surrogate of either pulmonary ventilation or blood circulation in the subject. Consequently, the computing device 120 can determine a respiration rate or heart rate for the accelerometer channel based on local maxima (peaks) and local minima (troughs) in the conditioned acceleration magnitude waveform. As an illustration, with reference to FIG. 2D, plot 240 presents an example of observed ECG signal for a subject in the bed 104 (FIG. 1) during the example time period. Further, plot 250 present the magnitude of accelerometer signal determined using the observed {aₓ}, {a_{y}}, and {a_{z}} during an example time interval. Peaks in plot 250 also are marked with crosses; respective triangles are also present to emphasize the peaks. As it can be gleaned from plot 250, peaks and troughs are more apparent, aligning better with the example ECG signal shown in plot 240. Similar to other diagrams, the example time interval shown in FIG. 2D is merely illustrative.

Referring again to FIG. 1, to identify peaks and troughs in a conditioned acceleration magnitude waveform, the computing device 120 can include an extrema identification module 134. The extrema identification module 134 can determine a peak by determining that the conditioned acceleration magnitude waveform exceeds a peak threshold value, and also determining that the conditioned acceleration magnitude waveform is a local maximum of such a waveform. In some cases, the extrema identification module 134 can update the peak threshold value after a current peak has been identified.

In addition or in some implementations, as part of determining the peak, the extrema identification module 134 can determine that a separation in time-domain between the local maximum and a second local maximum is equal to or greater than a first threshold time interval. The second local maximum immediately precedes the local maximum and may have been determined in a prior determination of a peak based on the conditioned acceleration magnitude waveform.

The extrema identification module 134 can determine a trough by determining that the conditioned acceleration magnitude waveform is less than a trough threshold value, and also determining that the conditioned acceleration magnitude waveform is a local minimum of such a waveform. In some cases, the extrema identification module 134 can update the trough threshold value after a current trough has been identified.

In addition or in some implementations, as part of determining the trough, the extrema identification module 134 can determine that a separation in time-domain between the local minimum and a second local minimum is equal to or greater than a second threshold time interval. The second local minimum immediately precedes the local minimum and may have been determined in a prior determination of a trough based on the conditioned acceleration waveform. In some cases, the second threshold time interval and the first threshold time interval can be equal to one another. In other cases, the second threshold time interval can be different from the first threshold time interval can be equal to one another.

Before identifying peaks and/or troughs for use in a determination of a vital sign (such as HR), the extrema identification module 134 can evaluate an adaptive window size w (see FIG. 2E, panel 260). Example principles of the adaptive window approach are delineated in panel 250 of FIG. 2E. More specifically, to identify peaks and troughs, extrema identification is done in a window of a particular size. Rather than configuring a fixed window size and using that size in the determination of peaks and troughs, the extrema identification module 134 can find a satisfactory window size in a set/interval [wₘᵢₙ, wₘₐₓ]. The satisfactory window size may be the best window size, the second window size, or the like. For each window size in that set/interval, the extrema identification module 134 can determine peaks and troughs in a conditioned acceleration magnitude signal. The extrema identification module 134 can determine the satisfactory window size (e.g., best window size) by solving a minimization problem with respect to the absolute difference between number of peaks and troughs across possible windows. That is, the satisfactory window size may be identified as the window size that minimizes |nₚₑₐₖₛ−n_{troughs}|, resulting in the number of peaks and troughs being the most balanced for the satisfactory window. An example implementation includes vectorizing the extrema identification problem by using a Toeplitz shift matrix.

After a peak has been identified in a conditioned acceleration magnitude waveform (or physiological signals), the extrema identification module 134 can update a peak dataset indicative of multiple peaks that have been detected up to a current time tₙ. The peak dataset includes timestamps corresponding to the measurement times of respective peaks. In some cases, the peak dataset is updated if the peak satisfies an inclusion rule. In one example, the inclusion rule dictates that in order to include a newly identified peak in the peak dataset, a separation in time-domain between the newly identified peak and an immediately prior peak must exceed a threshold time interval. The peak dataset can be part of data 154 retained in one or more memory devices 152 (collectively referred to as memory 152). The threshold time interval is configurable and can be based on the expected minimum RR and HR for an adult healthy subject (e.g. a human). In some implementations, the threshold time interval can be personalized to the subject being monitored, in case the subject has an underlying cardiologic condition, cardiopulmonary condition, particular physical attributes (e.g., the subject is an infant or a non-human animal), or the like.

Similarly, after a trough has been identified in the conditioned acceleration magnitude waveform (or physiological signals), the extrema identification module 134 can update a trough dataset indicative of multiple troughs that have been detected up to a current time tₙ. The peak dataset includes timestamps corresponding to the measurement times corresponding to respective peaks. In some cases, the trough dataset is updated if the trough satisfies an inclusion rule. In one example, the inclusion rule dictates that in order to include a newly identified trough in the trough dataset, a separation in time-domain between the newly identified trough and an immediately prior trough must exceed a threshold time interval. The trough dataset can be part of data 154 retained in the memory 152. The threshold time interval is configurable and can be based on the expected minimum RR and HR for an adult healthy human. In some implementations, the threshold time interval can be personalized to the subject being monitored, in case the subject has an underlying cardiologic condition, cardiopulmonary condition, particular physical attributes (e.g., the subject is an infant or a non-human animal), or the like.

The extrema identification module 134 can retain a peak dataset and a trough dataset within the memory 152, as part of multiple datasets 154.

To determine an estimate of a vital sign (e.g., HR or RR), the computing device 120 can include an estimator module 138. Such an estimate can be determined contemporaneously as accelerometer signals for respective channels become available. In other words, in sharp contrast to some existing, non-contact vitals monitoring technologies, the estimate quantifies the vital sign in real-time or nearly real-time.

As is illustrated in FIG. 1, the estimator module 138 can include an evaluator component 142 that can determine, using a peak dataset and/or trough dataset for a conditioned acceleration magnitude signal, one or more first estimates of a vital sign. To that end, the estimator module 138 can determine, using the peak dataset and the trough dataset, periods corresponding to respective consecutive peak-trough-peak triples. Such periods can be referred to as roundtrip peak time intervals. Each roundtrip peak time interval provides a particular heart rate (or, in some cases, a particular respiration rate).

The estimator module 138 can then provide a single estimate of the vital sign in real-time or essentially real-time. Such a practical application is absent from commonplace noncontact technologies. To provide a single estimate of a vital sign (e.g., HR), the estimator module 138 includes a merger component 144 that can combine multiple estimates of the vital sign (e.g., HR) in order to determine the single estimate of the vital sign (e.g., HR). Such a single estimate can be referred to as observed estimate.

The computing device 120, via the merger component 144, can combine multiple estimates arising from respective roundtrip peak time intervals within a defined sliding/moving time interval. In some cases, the computing device 120 combines the multiple estimates by averaging the multiple estimates over a sliding/moving time interval of 10 s. The computing device 120 assigns the average to a current observed estimate. Other sliding moving time intervals besides 10 s also can be implemented.

In response to determining a current observed estimate of a vital sign (e.g., RR or HR), the computing device 120 can cause a display device 160 to present the current observed estimate of the vital signal. Because the current observed estimate of the vital sign is part of a series of observed estimates of the vital sign, the computing device 120 can cause the display device 160 to present observed estimates of the vital sign at a defined presentation rate. The defined presentation rate is configurable. Examples of the defined presentation rate are 1 Hz and 5 Hz.

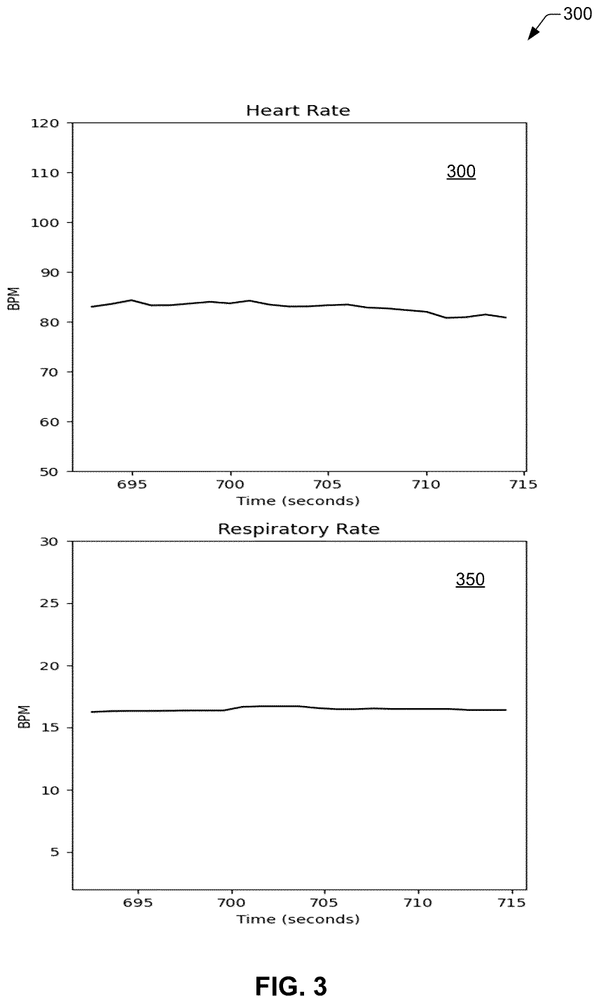

As illustrated in FIG. 1, the computing device 120 can include a reporting module 148. The reporting module 148 can cause the display device 160 to present observed estimates of a vital sign over time. To that end, the reporting module 148 can access a dataset containing a series of observed estimates of the vital sign to obtain an observed estimate of the vital sign. The reporting module 148 can access the dataset at the defined presentation rate. The reporting module 148 can then instruct the display device 160 to present the observed estimate of the vital sign. To that end, the reporting module 148 can send display data 156 to the display device 160. The display data 156 can include first data defining the observed estimate of the vital sign and, in some cases, second data defining formatting attribute(s) to present the observed estimate of the vital sign. The observed estimate of the vital sign can be presented in a graphical user interface (GUI) according to a defined format. The reporting module 148 can provide formatting information defining the visual appearance of the GUI. Simply as an illustration, FIG. 3 is an example of a GUI 300 where HR and RR are presented as a function of time, as the observed estimates of those vital signs become available in accordance with aspects described herein. The example GUI 300 incudes a first pane 300 presenting heart rate as a function of time, and a second pane 350 presenting respiration rate as a function of time.

As mentioned, the computing device 120 can retain observed HR estimates over time, as accelerometer signals become available. In some configurations, the computing device 120 can determine heart rate variability (HRV) using the observed HR estimates. To that end, in those configurations, the estimator module 138 can determine a difference AHR between a current observed HR estimate and an immediately prior observed HR estimate. The time difference between such consecutive heart beats can be determined, in units of seconds, as Δτ=60(Δ_{HR})−1. The estimator module 138 can retain the obtained current value of At in a dataset of the datasets 154 within the memory 152. As further accelerometer signals become available, the estimator module 138 can determine a series of values {Δτ} over a defined time interval ΔT (e.g., 180 s, 240 s, 300 s, or similar) and can retain the series of values {Δτ} in a particular dataset of the datasets 154. The estimator module 138 can then determine the variance of the series of values {Δτ} and can configure such a variance as an observed estimate of HRV for a subject being monitored. The reporting module 148, at the direction of the estimator module 138, for example, can send or otherwise made available the observed estimate of HRV for the subject. In some cases, the reporting module 148 can cause the display device to present the observed estimate of heart rate variability.

In some cases, the computing device 120 can supply observed estimates of a vital sign or several vital signs to a data storage platform (not depicted) remotely located relative to the computing device 120. For example, the reporting module 148 can cause the data storage platform (not depicted in FIG. 1) to retain observed estimates of a vital sign in a data repository containing medical record of the subject being monitored.

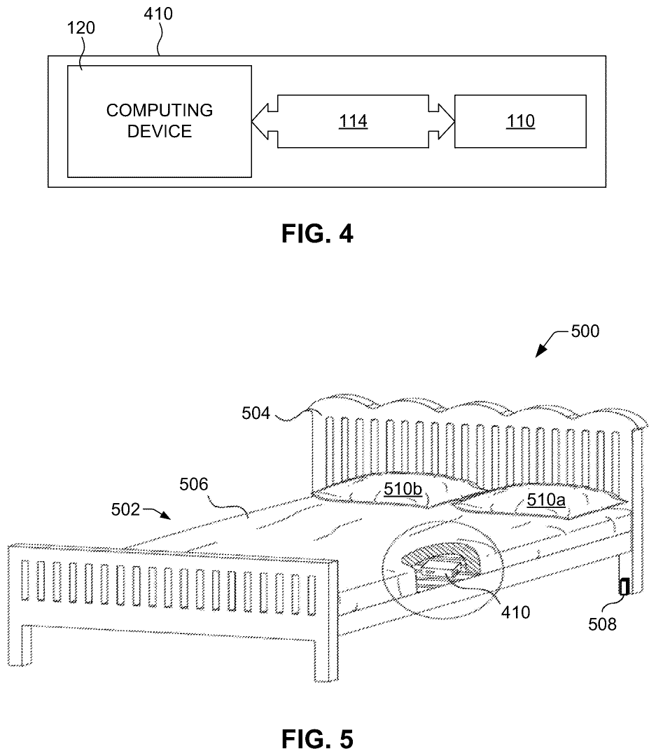

As it has been described, FIG. 1 illustrates an example system 100 having the accelerometer device 110 and the computing device 120 assembled at disparate locations, with only the accelerometer device 110 being integrated into the bed 104. The disclosure, of course, is not limited in that respect. Indeed, the systems for the contactless monitoring of vital signs in accordance with aspects of this disclosure can include a contactless monitoring unit that is integrated into a bed or another furniture and has enclosed therein the accelerometer device 110 and the computing device 120. To that point, simply as an illustration, FIG. 4 is a schematic block diagram of a contactless monitoring unit 410 that includes the computing device 120 and the accelerometer device 110 being functionally coupled with one another by the communication architecture 114.

FIG. 5 illustrates an example of an operating environment 500 for the contactless monitoring of vital signs, in accordance with one or more aspects of this disclosure. The operating environment 500 includes a bed 502 having a bed frame 504 and a mattress 506, and also includes the contactless monitoring unit 410 and a reference sensor device 508. The bed 502 can be an example of the bed 104 (FIG. 1). As is described herein, the operating environment 500 includes a setup for a subject (e.g., a patient) to recline or rest in a supine, prone, right/left lateral recumbent, seated, or other various positions on the mattress 506. In some examples, the contactless monitoring unit 410 is embedded within the mattress 506. In other examples, the contactless monitoring unit 410 is embedded within a pillow 510a or a pillow 510b. In some examples, the contactless monitoring unit 410 is positioned proximate the mattress (e.g., on or within the bed frame 504; not shown). The reference sensor system 508 is positioned on (or within) the bed frame 504 and can sense noises and vibrations that are external to the mattress 506 and do not originate from the mattress 506. For example, the reference sensor system 508 senses vibrations and interference that does not originate from the subject being monitored.

In some embodiments, one or more accelerometer devices are configured at a specified location on the bed 502. In one configuration, two accelerometer devices can be assembled on top of the mattress 506, with one accelerometer device close to a location where a subject would lie and another accelerometer on the ground. The accelerometer device on the ground can generate accelerometer signals from other equipment (e.g., a refrigerator, HVAC equipment, or similar) in the space where the bed 502 is located. Thus, the accelerometer device on the ground can be used to remove environment noise from the accelerometer signals from the accelerometer device(s) in assembled in the bed 502. In some cases, additional sensor devices and accelerometer devices may be placed proximate the mattress 506 in various configurations. For example, an additional accelerometer device may be placed beside the subject within a first range (e.g., up to 15 cm away from the subject) primarily for noise filtration. The accelerometer data from the one or more accelerometer devices can be used to filter out outside vibrations (e.g., noise) by aggregating datasets from the one or more accelerometer devices. After acceleration signals are conditioned in accordance with aspects described herein, the physiological signal can be recovered.

In some cases, the configuration of the one or more accelerometer devices includes mounting the one or more accelerometer devices to a rigid plate to reduce noise and vibrations. One or more additional accelerometer devices can be used to filter out additional external noise (e.g., walking, clapping, and/or shouting sounds). The one or more additional accelerometer devices can be useful to enhance the signal quality and to improve fidelity of the tracked accelerometer signals.

In some implementations, signal from a reference sensor device 508 is used to filter out external noise. Vibrations originating from outside of the mattress 506 or bed frame 504 propagate into them and can introduce significant noise into the acceleration signals measured by an accelerometer device integrated into the contactless monitoring unit 410. The vibrations can be caused by various external sources, such as footsteps in a space that contains the bed 502 or a vehicle passing outside such a space. This can disrupt the contactless monitoring techniques implemented by the contactless monitoring unit 410 and also can corrupt the output of such techniques. In some cases, the external vibrations can be mitigated by placing a reference sensor device 508 on the floor next to one of the bed posts or on a leg of the bedframe 504 itself. This reference sensor device 508 measures vibration signals in the same way as the accelerometer device(s) integrated into the contactless monitoring unit 410. However, measurements generated by the reference sensor device 508 can exclude the vital signs because of the distance that separates the reference sensor device 508 and the subject being monitored.

The contactless monitoring unit 410 can include one or more low-noise accelerometer devices. The contactless monitoring unit 410 also can include one or more electronic devices having a processor configured to receive, process, transmit, and/or analyze data from the one or more low noise accelerometer devices. The one or more accelerometer devices can be configured on a rigid structure and placed in distinct locations with respect to the other one or more accelerometer devices. The mechanical movements of the heart can be detected by one or more accelerometer devices placed within a reasonable distance of the subject being monitored, without the subject being in direct contact with the contactless monitoring unit 410. Each one of the accelerometer device(s) are coupled to a processor to ensure that raw acceleration signals are timestamped and recorded.

The contactless monitoring unit 410 can include one or more accelerometer devices functionally coupled (e.g., mechanically, electrically, and/or communicatively coupled) with a microcontroller. One or many insulation-displacement contact (IDC) connectors can provide such functional coupling.

In some embodiments, the contactless monitoring unit 410 may be coupled to various external devices for further analysis and/or distribution. In some examples, the contactless monitoring unit 410 may include one or more cables coupling the monitoring system to the various external devices. In some examples, the contactless monitoring unit 410 wirelessly communicates with one or more external devices.

Additional considerations include introducing a rigid plastic plate beneath one or more of the accelerometers to house the accelerometer, signal processing and data transmission device to increase the sensitivity. This can be achieved by physically affixing the housing to the frame of a chair and/or a bed.

In some cases, a second contactless vital signal monitoring system is placed on the other side of the bed 502 to measure vital signs from a second subject. When multiple subjects are laying on the bed 502, additional processing may be required to differentiate between their physiological signals as observed by an in-bed sensor. To account for this, a system is provided in which multiple sensors are placed on or in the bed and algorithmic processing is applied to separate the signals corresponding to the occupants. This enables multi-point sensing and reporting of vital signs for each subject. In various examples, the contactless monitoring unit 410 receives second signals recorded by the second contactless vital sign monitoring system and uses the second signals to filter out noise and interference from the signals measured at the contactless monitoring unit 410. The algorithmic processing can include one or more of several multi-channel techniques including beamforming and/or source separation. Beamforming is a well-known technique in array signal processing. Source separation is often used in the audio signal processing.

In some cases, the contactless monitoring unit 410 can determine an orientation of subject with respect to the bed. An adult subject is usually oriented longitudinally along the length of the bed. In contrast, in infant care, the orientation of the subject can be inconsistent or a priori unknown. Using the contactless monitoring unit 410, the orientation of a subject can be determined from data collected by an accelerometer device by analyzing signals in the x-y plane of the accelerometer device in the frequency range occupied by heart-related vibrations, for example. A subject's heart naturally pumps blood upwards in the direction of the head, which causes a recoil force towards the subject's feet. This causes a major axis of vibration to emerge in the recorded signals that can be detected with principal component analysis or similar techniques.

In some cases, respiratory signals extracted from accelerometer signals can be used to identify patterns of breathing indicative of respiratory conditions such as obstructive sleep apnea. Sleep apnea is characterized by occasional cessation of breathing for at least several seconds. Counting the number of such events during a subject's night's sleep and dividing by the duration of sleep gives the apnea-hypopnea index (AHI), a commonly used metric to quantify severity of apnea. For the specific case of sleep apnea, the respiration signal is extracted, and periods of cessation of breathing are identified and counted.

In some scenarios in accordance with this disclosure, the approaches used for contactless monitoring of vital signs of a subject may be implemented in a contact modality, using a wearable device. An example of the wearable device is a patch device that can be adhered or otherwise affixed to the subject.

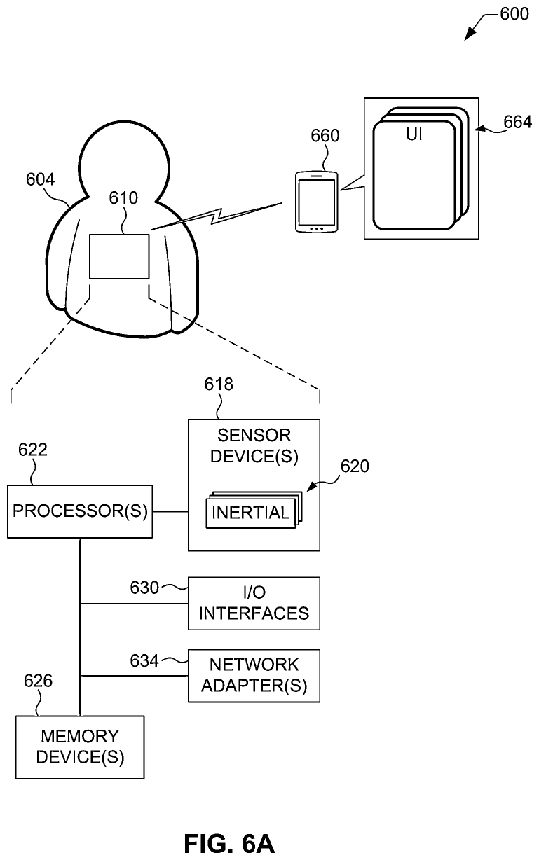

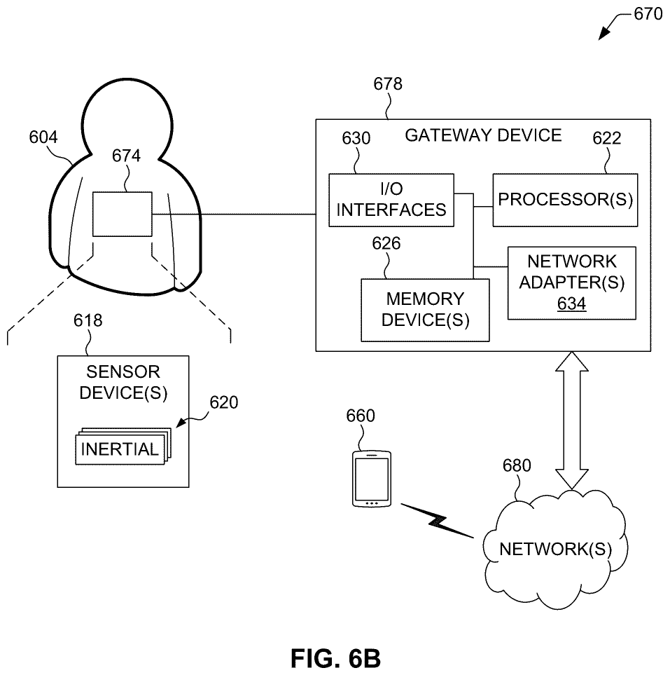

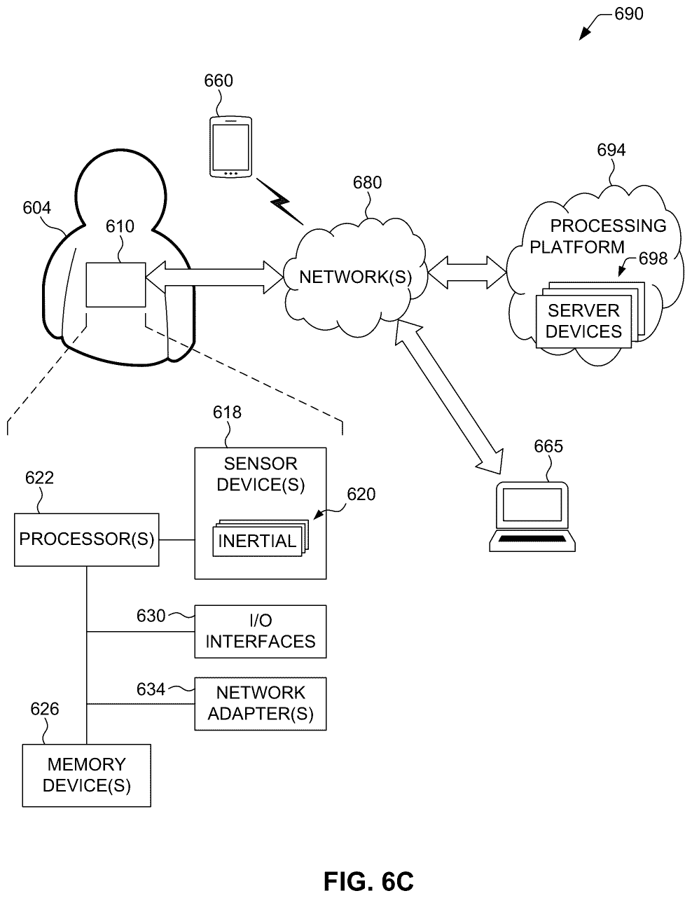

In some cases, as is illustrated in the example system 600 shown in FIG. 6A, a wearable device 610 can be affixed to a subject 604. The wearable device 610 includes one or more sensor devices 618. The sensor device(s) 618 can include one or more inertial devices 620. The wearable device 610 can be mounted on the torso of the subject 604 such that each inertial sensor device is mechanically coupled to the chest of the subject 604. Specifically, each inertial sensor can be in contact with a solid medium that, in turn, is in contact with the chest of the subject 604. In some configuration, a single inertial sensor device is assembled in the wearable device 610. As an example, the single inertial sensor device can include a three-axis low-noise accelerometer. For instance, the single inertial sensor device can be the accelerometer device 110 described herein. In other configurations, the inertial sensor device(s) 620 can include multiple inertial sensor devices, e.g., a three-axis accelerometer and a three-axis gyroscope. The wearable device 610 also includes one or more processors 622, one or more memory devices 626, multiple

I/O interfaces 630, and one or more network adapters 634. At least one of the processor(s) 622 and at least one of the memory device(s) 626 can form a control unit. In some cases, the control unit can implement control operations involved in setup/placement of the wearable device 610 and/or performance of one or more measurements by at least one of the multiple sensor devices 618, resulting in measurement signals. In addition, or in some configurations, the processor(s) 622 can implement various of the approaches described herein. For example, the processor(s) 622 can implement the techniques described herein for monitoring of vital signs. To that end, at least one of the processor(s) 622 can execute processor-accessible instructions (e.g., processor-readable instructions and/or processor executable instructions) retained in the memory device(s) 626. That is, in some cases, the computing resources that are present in the wearable device 610 permits the wearable device to operate as the computing device 120 described herein. More specifically, the processor(s) 622 can implement the techniques described herein for monitoring of vital signs. To that point, at least one of the processor(s) 622 can execute processor-accessible instructions (e.g., processor-readable instructions and/or processor-executable instructions) retained in the memory device(s) 626. For example, in some cases, the processor-accessible instructions can form or can be part of the intake module 122, the conditioning module 126, the validation module 130, the extrema identification module 134, the estimator module 138, and the reporting module 148 described herein.

In some configurations, the wearable device 610 also can analyze the data indicative of the vital sign(s) that the wearable device 610 can generate. The analysis can be directed to determining if one or more of the vital sign(s) have values within a desired range. The desired range may be a range considered clinically normal or a target range personalized for the subject 604. The wearable device 610 can perform one or more actions in response to a value of a vital sign or a group of values of the vital sign being outside the desired range. The action(s) can include presenting an audible alarm, presenting a visible alarm (e.g., causing one or more light sources, such as LEDs of a particular color to be energized steadily or alternately), presenting a mechanical alarm by causing a haptic device to be energize and create a vibration or other movement, or similar alarms.

A first network adapter of the network adapter(s) 634 can be embodied in a radio unit that permits exchanging information wirelessly between the wearable device 610 and a user device 660 (or another type of device). The radio unit can operate according to various radio technologies. In one example, the radio unit can permit the wireless exchange of information according to Bluetooth protocols for wireless communication. The user device 660 can be owned, leased, and/or operated by the subject 604. An aid assisting the subject with performing measurements also can operate the user device 660. The aid can be, for example, a family member, a nurse assistant, nursing home staff, or similar support personnel.

The user device 660 can be configured with a software application, or another type of program code, to exchange information the wearable device 610. The user device 660, via the software application configured in the user device 660, can receive such data indicative of one or more vital signs (e.g., HR, RR, or both) and/or instructions to present such data. The user device 660 can present that data in at least one of multiple user interfaces 664 that the user device 660 can present in response to the software application being executed. An example of a user interface presented by the user device 660 is the GUI 300 (FIG. 3).

In some configurations, the user device 660 can analyze the data indicative of the vital sign(s) to determine if one or more of the vital sign(s) have values within a desired range. The desired range may be a range considered clinically normal or a target range personalized for the subject 604. The user device 660, or any other device functionally coupled with the wearable device 610 and performing such an analysis, can perform one or more actions in response to a value or group of values being outside the desired range. The action(s) can include presenting an audible alarm, a visible alarm, sending a message to a designated recipient (clinician, aid, family member), presenting a message (e.g., a pop-up message), causing the wearable device 610 to energize one or more light sources (e.g., LEDs of a particular color), or the like. The light source(s) can be energized for a first time interval, turned off for a second time interval, and energized again for the first time interval, and repeating that cycle to cause the light source(s) to blink.

In some cases, in addition to or instead of presenting data indicative of a vital sign in a user interface, the wearable device 610 can include and can use one or more lighting devices (e.g., one or more LEDs) within the I/O interfaces 630 to present or otherwise convey values of a vital sign (e.g., HR or RR). For example, lighting device(s) configured to emit light of a first color may be used to communicate if the HR/RR is outside a safe zone, second lighting device(s) configured to emit light of a second color may be used to communicate if the HR/RR is slightly outside (within a defined range or a tolerance from) an expected range, and third lighting device(s) configured to emit light of a third color may be used to communicate if the HR/RR is in an unsafe range. In addition or in some cases, blinking speed is another way to convey values of vital signs obtained using accelerometer signals, in accordance with aspects described herein. Further or in other cases, the wearable device 610 also may include a haptic device that may be energized in situations when a vital sign is in an unsafe range. The haptic device may also be energized, with a different amplitude of vibration, for example, to convey that a vital sign in slight outside range.

As is illustrated in the example system 670 shown in FIG. 6B, the determination of the vital signs can be implemented externally to a wearable device 674 (e.g., a patch device or another type of wearable device that can be mounted to the chest or torso of the subject 604). In the example system 670, the architecture of the wearable device 674 may be simplified relative to the architecture of the wearable device 610 in the example system 600 (FIG. 6A). Specifically, the wearable device 674 can have limited computational resources (not depicted in FIG. 6B) and can include the one or more inertial sensors 620. Such inertial sensor(s) can provide accelerometer signals for x, y, and z channels that may be used to determine vital signs in accordance with aspects described herein. The disclosure is not limited to the wearable device 674 being simpler than the wearable device 610 shown in FIG. 6A. Indeed, in some cases, the wearable device 674 and the wearable device 610 may have similar or same computing resources.

The wearable device 674 can supply accelerometer signals generated by at least one of the inertial sensor(s) 620 to a gateway device 678. The that end, the wearable device 674 can be communicatively coupled with the gateway device 678 wirelessly or via a wireline connection (both schematically depicted by a straight line in FIG. 6B). The gateway device 678 can be coupled with the wearable device in such fashion via the I/O interface(s) 630 and/or the network adapter(s) 634.

The gateway device 678 can implement various of the approaches described herein to determine vital sign(s) of the subject 604 using the accelerometer signals received from the wearable device 674. As is described herein, an example of the vital sign(s) is RR and another example of the vital sign(s) is HR. To that end, the gateway device 6748 can include the processor(s) 622 and the memory device(s) 626. As mentioned, the processor(s) 622 can implement the techniques described herein for monitoring of vital signs. To that point, at least one of the processor(s) 622 can execute processor-accessible instructions (e.g., processor-readable instructions and/or processor-executable instructions) retained in the memory device(s) 626. For example, in some cases, the processor-accessible instructions can form or can be part of the intake module 122, the conditioning module 126, the validation module 130, the extrema identification module 134, the estimator module 138, and the reporting module 148 described herein.

The gateway device 678 also can be communicatively coupled with the user device 660 via at least one of network(s) 680 (e.g., wireless network(s), wireline network(s), or a combination of both types of networks). The gateway device 678 can send data indicative of vital signs (e.g., HR, RR, or both) of the subject 604. The user device 660, via the software application configured in the user device 660, can receive such data and/or instructions to present such data. As is described herein, the user device 660 can present the data in one or more user interfaces.

In addition or in other cases, as is illustrated in example system 690 shown in FIG. 6C, the wearable device 610 can be communicatively coupled with a processing platform 694 via the network(s) 680 and can provide data to the processing platform 694. The data can be indicative of indicative of accelerometer signals generated by the inertial sensor device(s) 620. The processing platform 694 can include multiple server devices 698. At least one of the server devices 698 can implement various of the approaches described herein to determine vital sign(s) of the subject 604 using the accelerometer signals received from the wearable device 610. To that end, the server device can include processor(s) and memory device(s). The processor(s) can implement the techniques described herein for monitoring of vital signs.

Rather than being directly communicatively coupled with the user device 660, as is the case in the example system 600 (FIG. 6A), the wearable device 610 in the example system 690 (FIG. 6C) can be functionally coupled with the user device 660 via at least one network of the network(s) 680. Regardless of the particular type of communicative coupling, the user device 660 (in example system 600 or example system 690), via the software application configured in the user device 660, can receive data indicative of one or more vital signs (e.g., HR, RR, or both) and/or instructions to present such data. The user device 660 can present the data in at least one of multiple user interfaces that the user device 660 can present in response to the software application being executed.

The example system 690 also may include a computing device 665 that can be remotely located relative to both the processing platform 694 and a location of the subject 604. A locale of the computing device 665 can be a healthcare facility or a dwelling of an operator of the computing device 665, for example. The computing device 665 can be mobile in some cases, and thus, the locale can change over time as the computing device 665 moves from one location to another. The computing device 665 is communicatively coupled with the processing platform 694 via at least one network of the network(s) 680. The computing device 665 may be associated with, or otherwise operated by, a clinician or, in some cases, a veterinarian. In other cases, another computing device (not shown in FIG. 6C) also can be functionally coupled with the processing platform 694. That other computing device may be associated with, or otherwise operated by, an agent of a health insurance provider. The agent may be a human or an autonomous bot executed by such a computing device. The clinician (or, in some cases) the veterinarian) may be involved in providing care for the subject 604. The agent may be involved in making decisions related to financial coverage of such care.

It is noted that while not shown in FIG. 6B, the example system 670 also can include the computing device 665 described hereinbefore.

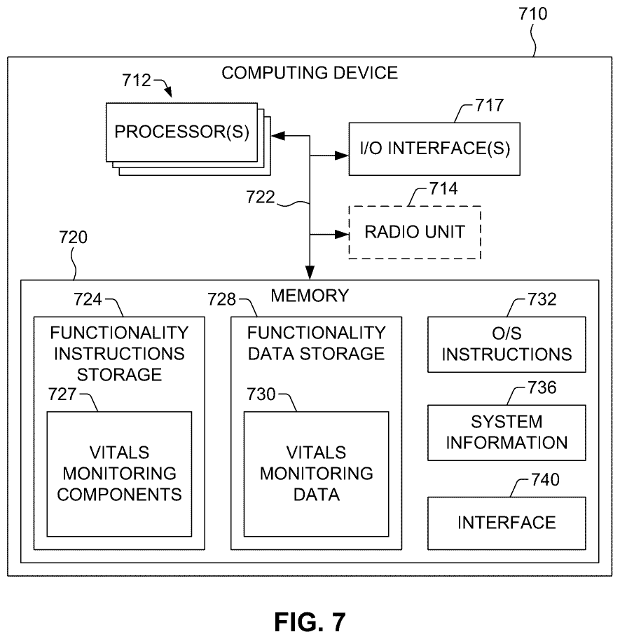

FIG. 7 illustrates an example of a computing device 710 that can provide various functionalities of contactless monitoring of vital signs in accordance with aspects of this disclosure. The computing device 710 can embody the computing device 120 (FIG. 1). The computing device 710 can provide such functionalities in response to execution of one or more software components retained within the computing device 710. Such component(s) can render the computing device 710 a particular machine for contactless monitoring of vital signs, among other functional purposes that the device 710 may have. A software component can be embodied in or can comprise one or more processor-accessible instructions, e.g., processor-readable and/or processor-executable instructions. In one scenario, at least a portion of the processor-accessible instructions can embody and/or can be executed to perform at least a part of one or more of the techniques described herein. The one or more processor-accessible instructions that embody a software component can be arranged into one or more program modules, for example, that can be compiled, linked, and/or executed at the computing device 710 or other computing devices. Generally, such program modules comprise computer code, routines, programs, objects, components, information structures (e.g., data structures and/or metadata structures), etc., that can perform particular tasks (e.g., one or more operations) in response to execution by one or more processors 712 integrated into the computing device 710.

The various example aspects of the disclosure can be operational with numerous other general purpose or special purpose computing system environments or configurations. Examples of well-known computing systems, environments, and/or configurations that can be suitable for implementation of various aspects of the disclosure in connection contactless monitoring of vital signs can include personal computers; server computers; laptop devices; handheld computing devices, such as mobile tablets or laptop computers; wearable computing devices; and multiprocessor systems. Additional examples can include network personal computers (PCs), mainframe computers, blade computers, programmable logic controllers, embedded systems, distributed computing environments that comprise any of the above systems or devices, and the like.

As is illustrated in FIG. 7, the computing device 710 includes one or multiple processors 712, one or multiple input/output (I/O) interfaces 717, one or more memory devices 720 (referred to as memory 720), and a bus architecture 722 (referred to as bus 722) that functionally couples various functional elements of the computing device 710. The computing device 710 can include, optionally, a radio unit 714. The radio unit 714 can include one or more antennas and a communication processing unit that can permit wireless communication between the computing device 710 and another device, such as a remote computing device, a display device, and/or a remote sensor device. The bus 722 can include at least one of a system bus, a memory bus, an address bus, or a message bus, and can permit the exchange of information (data and/or signaling) between the processor(s) 712, the I/O interface(s) 717, and/or the memory 720, or respective functional elements therein. In some cases, the bus 722 in conjunction with one or more internal programming interfaces 740 (also referred to as interface 740) can permit such exchange of information. In cases where the processor(s) 712 include multiple processors, the computing device 712 can utilize parallel computing.

The I/O interface(s) 717 can permit communication of information between the computing device 710 and an external device, such as another computing device, a display device, or similar device. Such communication can include direct communication or indirect communication, such as the exchange of information between the computing device 710 and the external device via a network or elements thereof. As illustrated, the I/O interface(s) 717 can include one or more of network adapter(s), peripheral adapter(s), and display unit(s). Such adapter(s) can permit or otherwise facilitate connectivity between the external device and one or more of the processor(s) 712 or the memory 720. For example, the peripheral adapter(s) can include a group of ports, which can include at least one of parallel ports, serial ports, Ethernet ports, V.35 ports, or X.21 ports. In certain aspects, the parallel ports can comprise General Purpose Interface Bus (GPIB), IEEE-1284, while the serial ports can include Recommended Standard (RS)-232, V.11, Universal Serial Bus (USB), FireWire or IEEE-1394.

The I/O interface(s) 717 can include a network adapter that can functionally couple the computing device 710 to one or more remote computing devices, display devices, or sensors devices (not depicted in FIG. 7) via one or more traffic and signaling pipes that can permit or otherwise facilitate the exchange of traffic and/or signaling between the computing device 710 and such one or more remote computing devices or sensors. Such network coupling provided at least in part by the network adapter can be implemented in a wired environment, a wireless environment, or both. The information that is communicated by the network adapter can result from the implementation of one or more operations of a method in accordance with aspects of this disclosure. The I/O interface(s) 717 can include more than one network adapter in some cases.

In addition, or in some cases, depending on the architectural complexity and/or form factor the computing device 710, the I/O interface(s) 717 can include a user-device interface unit that can permit control of the operation of the device 710, or can permit conveying or revealing the operational conditions of the computing device 710. The user-device interface can be embodied in, or can include, a display unit. The display unit can include a display device that, in some cases, has touch-screen functionality. In addition, or in some cases, the display unit can include lights, such as light-emitting diodes, that can convey an operational state of the computing device 710.

The bus 722 can have at least one of several types of bus structures, depending on the architectural complexity and/or form factor the computing device 710. The bus structures can include a memory bus or a memory controller, a peripheral bus, an accelerated graphics port, and a processor or local bus using any of a variety of bus architectures. As an illustration, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, a Peripheral Component Interconnect (PCI) bus, a PCI-Express bus, a Personal Computer Memory Card International Association (PCMCIA) bus, a Universal Serial Bus (USB), and the like.

The computing device 710 can include a variety of computer-readable media. Computer-readable media can be any available media (transitory and non-transitory) that can be accessed by a computing device. In one aspect, computer-readable media can comprise computer non-transitory storage media (or computer-readable non-transitory storage media) and communications media. Examples of computer-readable non-transitory storage media include any available media that can be accessed by the computing device 710, including both volatile media and non-volatile media, and removable and/or non-removable media. The memory 720 can include computer-readable media in the form of volatile memory, such as random access memory (RAM), and/or non-volatile memory, such as read-only memory (ROM).

The memory 720 can include functionality instructions storage 724 and functionality data storage 728. The functionality instructions storage 724 can include computer-accessible instructions that, in response to execution (by at least one of the processor(s) 712, for example), can implement one or more of the functionalities of this disclosure in connection with keyphrase detection. The computer-accessible instructions can embody, or can include, one or more software components illustrated as vitals monitoring component(s) 727. Execution of at least one component of the vitals monitoring components 727 can implement one or more of the methods described herein. Such execution can cause a processor (e.g., one of the processor(s)) that executes the at least one component to carry out at least a portion of the methods disclosed herein. In some cases, the vitals monitoring component(s) 727 can include the intake module 122, the conditioning module 126, the validation module 130, the extrema identification module 134, the estimator module 138, and the reporting module 148.

A processor of the processor(s) 712 that executes at least one of the vitals monitoring components 727 can retrieve data from or retain data in one or more memory elements 720 in the functionality data storage 728 in order to operate in accordance with the functionality programmed or otherwise configured by the vitals monitoring components 727. The one or more memory elements 730 may be referred to as vitals monitoring data 730. Such information can include at least one of code instructions, data structures, or similar. For example, at least a portion of such data structures can define the datasets 154 (FIG. 1) and, thus, can define time series of acceleration values for various accelerometer channels, time series of vital sign values, time series of HRV values, and/or any other type of data relevant to contactless monitoring vital signs in accordance with aspects of this disclosure.

The interface 740 (e.g., an application programming interface) can permit or facilitate communication of data between two or more components within the functionality instructions storage 724. The data that can be communicated by the interface 740 can result from implementation of one or more operations in a method of this disclosure. In some cases, one or more of the functionality instructions storage 724 or the functionality data storage 728 can be embodied in or can comprise removable/non-removable, and/or volatile/non-volatile computer storage media.

At least a portion of at least one of the vitals monitoring components 726 or the vitals monitoring data 730 can program or otherwise configure one or more of the processor(s) 712 to operate at least in accordance with the functionality described herein. One or more of the processor(s) 712 can execute at least one of the vitals monitoring components 727, and also can use at least a portion of the data in the functionality data storage 728 in order to provide contactless monitoring of vital signs in accordance with one or more aspects described herein. In some cases, the functionality instructions storage 724 can embody or can include a computer-readable non-transitory storage medium having computer-accessible instructions that, in response to execution, cause at least one processor (e.g., one or more of the processor(s) 712) to perform a group of operations comprising the operations or blocks described in connection with example methods disclosed herein.

In addition, the memory 720 can include processor-accessible instructions and information (e.g., data, metadata, and/or program code) that permit or facilitate the operation and/or administration (e.g., upgrades, software installation, any other configuration, or the like) of the computing device 710. Accordingly, as illustrated, the memory 720 can include a memory element 732 (labeled operating system (O/S) instructions 732) that contains one or more program modules that embody or include one or more operating systems, such as Windows operating system, Unix, Linux, Symbian, Android, Chromium, and substantially any OS suitable for mobile computing devices or tethered computing devices. In one aspect, the operational and/or architectural complexity of the computing device 710 can dictate a suitable O/S. The memory 720 also includes system information storage 736 having data, metadata, and/or program code that permits or facilitates the operation and/or administration of the computing device 710. Elements of the O/S instructions 732 and the system information storage 736 can be accessible or can be operated on by at least one of the processor(s) 712.

It should be recognized that while the components retained in the functionality instructions storage 724 and other executable program components, such as the O/S instructions 732, are illustrated herein as discrete blocks, such software components can reside at various times in different memory components of the computing device 710, and can be executed by at least one of the processor(s) 712.

The computing device 710 can include a power supply (not shown), which can power up components or functional elements within such devices. The power supply can be a rechargeable power supply, e.g., a rechargeable battery, and it can include one or more transformers to achieve a power level suitable for the operation of the computing device 710 and components, functional elements, and related circuitry therein. In some cases, the power supply can be attached to a conventional power grid to recharge and ensure that such devices can be operational. To that end, the power supply can include an I/O interface (e.g., one of the interface(s) 717) to connect to the conventional power grid. In addition, or in other cases, the power supply can include an energy conversion component, such as a solar panel, to provide additional or alternative power resources or autonomy for the computing device 710.

In some scenarios, the computing device 710 can operate in a networked environment by utilizing connections to one or more remote computing devices and/or sensor devices (not depicted in FIG. 7). As an illustration, a remote computing device can be a personal computer, a portable computer, a server, a router, a network computer, a peer device or other common network node, and so on. As described herein, connections (physical and/or logical) between the computing device 710 and a remote computing device or sensor can be made via one or more traffic and signaling pipes, which can comprise wired link(s) and/or wireless link(s) and several network elements (such as routers or switches, concentrators, servers, and the like) that form a local area network (LAN), a wide area network (WAN), and/or other networks (wireless or wired) having different footprints.

One or more of the techniques disclosed herein can be practiced in distributed computing environments, such as grid-based environments, where tasks can be performed by remote processing devices (e.g., network servers) that are functionally coupled (e.g., communicatively linked or otherwise coupled) through a network having traffic and signaling pipes and related network elements. In a distributed computing environment, one or more software components (such as program modules) may be located in both the computing device 710 and at least one remote computing device.

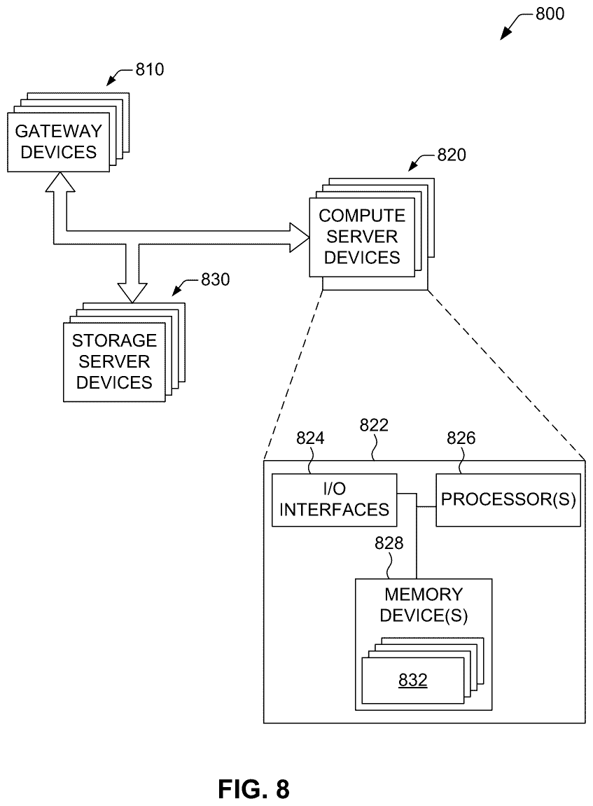

FIG. 8 is a schematic block diagram of an example of a computing system 800 that can determine vital signs of subject using accelerometer signals, in accordance with one or more embodiments of this disclosure. The accelerometer signals can be generated by an inertial sensor device (such as the accelerometer device 110). The computing system 800 can embody, or can be part of, the processing platform 694 described herein in connection with example systems of this disclosure. Accordingly, the example computing system 800 can provide at least some of the functionality described herein in connection with monitoring vital signs using accelerometer signals in accordance with aspects described herein.

As mentioned, the processing platform 694 includes multiple server devices 698. The multiple server devices 698 can be arranged in a cloud architecture. As such, the example computing system 800 includes two types of server devices: Compute server devices 820 and storage server devices 830. A subset of the compute server devices 820, individually or collectively, can host various modules 832 that permit implementing the approaches described herein in connection with monitoring vital signs using accelerometer signals in accordance with aspects described herein. Thus, the subset of compute server devices 820 can operate in accordance with functionality described herein in connection with monitoring vital signs using accelerometer signals as is described herein.

The architecture of each of the compute server devices 820, including the compute server device 822, includes multiple input/output (I/O) interfaces 824, one or more processors 826, one or more memory devices 828, and a bus architecture that functionally couples the processor(s) and the memory device(s). In some cases, the compute server device 822 can store the modules 832 in at least one of such memory device(s) 828. At least the subset of the compute server devices 820 can be functionally coupled to one or multiple ones of the storage server devices 830. The coupling can be direct or can be mediated by at least one of the gateway devices 810. The storage server devices 830 include data and/or metadata that can be used to implement the functionality described herein in connection with monitoring vital signs using accelerometer signals as is described herein.

Each one of the gateway devices 810 can include one or multiple processors functionally coupled to one or multiple memory devices that can retain application programming interfaces (APIs) and/or other types of program code for access to the compute server devices 820 and storage server devices 830. Such access can be programmatic, via a defined function call, for example. The subset of the compute server devices 820 that host one or a combination of modules that can use API(s) supplied by the gateway devices 810 in order to provide results of implementing the functionalities described herein in connection with monitoring of vital signs using accelerometer signals in accordance with aspects described herein.

Acceleration signals generated by the accelerometer device 110 (FIG. 1) or another inertial sensor device can be used to determine presence or absence of a subject in a bed that includes or is otherwise coupled with the accelerometer device 110 or the inertial sensor device. A determination that a subject is present in the bed-that is, detection of the subject in the bed-can permit implementing numerous actions, such as initiating monitoring of vital signs of the subject. Simply for purposes of illustration, by determining the presence or absence of the subject in the bed, a computing device (computing device 120 (FIG. 1) or user device 660, for example) can determine whether to suppress or output estimates of vital signs (HR or RR, or both) at a display device (e.g., display device 160 (FIG. 1)) or another type of device that permits visualizing or otherwise consuming data indicative of vital signs of the subject. Specifically, the computing device can cause the display device or the other device to present estimates of vital signs when a subject is actually in the bed. In situations when a subject is not in the bed, the computing device can either terminate determining estimates of vital signs and/or can cause a display device not to output data indicative of estimates of vital signs.

One of many benefits of determining presence or absence of a subject in bed to control the supply and/or presentation of data indicative of vital sign estimates (e.g., estimates of HR and/or RR) is that monitoring vital signs as is described herein can be used to detect vitals outside a normal range (or, in some cases, a target range) and in response, notify a clinician, veterinarian, or another type of caregiver. Supplying spurious vital sign data when a subject is absent from the bed may lead to false alarms of vitals being out of range, since the vitals were generated on noise.

Because the subject may be a pet or another type of mammal (such as a human), determining presence or absence of that type of subject may cause the implementation of particular actions. For example, in a scenario where a pet or human is being hospitalized, a determination of the pet or human being absent from the bed may cause a device to present an audible and/or visible alarm. Such an alarm can prompt a caregiver to return the pet or human to the bed. Such a device can be controlled in such a fashion by the computing device that implements the approach to detect a subject in bed as is described herein.

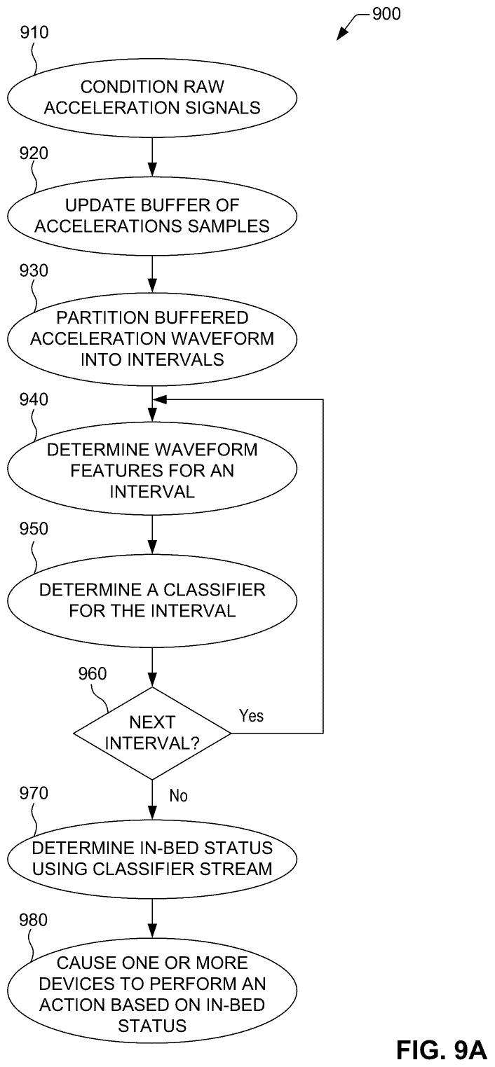

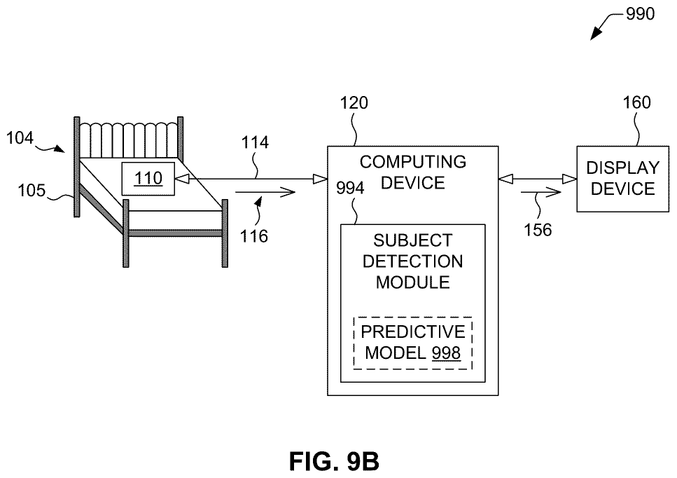

FIG. 9A illustrates an example of a process flow for determining presence or absence of a subject in a bed, in accordance with one or more aspects of this disclosure. A computing device or system of computing devices can implement the example approach 900. To that end, each computing device includes computing resources that may implement at least one of the stages included in the example process flow 900 and other methods described herein. The computing resources comprise, for example, central processing units (CPUs), graphics processing units (GPUs), tensor processing units (TPUs), memory, disk space, incoming bandwidth, and/or outgoing bandwidth, interface(s) (such as 1/0 interfaces or APls, or both); controller devices(s); power supplies; a combination of the foregoing; and/or similar resources. As such, each computing device can one or more modules, and can implement the example process flow 900 by executing the module(s), individually or in combination. In some cases, the computing device 710 (FIG. 7) may implement the example process 900. In other cases, the computing system 800 (FIG. 8) may implemented the process flow 900. Simply as an example, the computing device 120 that is included in the example system 990 (FIG. 9B) can implement the example process flow 900. To that end, the computing device 120 may include a subject detection module 994. In some cases, the example system 990 can include a second computing device (not depicted in FIG. 9B) that can include the subject detection module 994 and can implement the example process flow 990. In some embodiments, the subject detection module 994 can be part of vitals monitoring components 727 (FIG. 7).

Back to referring to FIG. 9, at stage 910, the computing device(s), individually or in combination, can condition raw (or source) acceleration signals. The computing device(s) can condition the raw acceleration signals via the conditioning module 126, for example. The raw acceleration signals correspond to respective channels in the accelerometer device 110 (FIG. 1 or FIG. 9B) or the inertial sensor device. The computing device(s) can receive a raw acceleration signal as chunk of acceleration samples at a time; that is, a defined number of acceleration samples at a time. Chunks may be received periodically, according to a schedule, or as the chunk becomes available at the accelerometer device 110 or the inertial sensor device. Operations included in conditioning the raw acceleration signals may include, for example, converting raw accelerometer signals from units of milli-g to SI units (that is, m/s²), resulting in a waveform representing an acceleration vector a_{SI} at each sample. The operations also may include subtracting, at each sample, the expected gravity vector (0,0, g) from the a_{SI}, resulting in aᵣₑₗ=a_{SI}−(0,0, g). Such a conditioning operation causes the accelerometer signal in each channel x, y, and z to have similar amplitude. The operations may then include determining magnitude of accelerometer signal: a_{mag}=∥aᵣₑₗ∥. The magnitude of accelerometer signal is a time series of values (see diagram 250 in FIG. 2D, for example) defining a magnitude signal. The operations may further include detrending the magnitude signal. The magnitude signal may be detrended, in some cases, by applying a first order IIR high-pass filter to the magnitude signal.

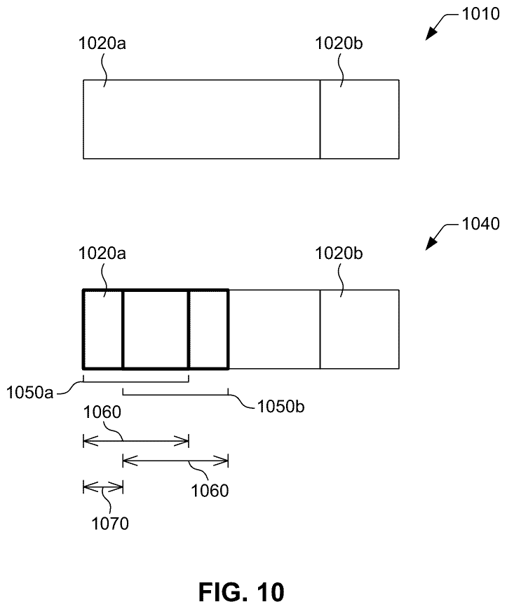

At stage 920, the computing device(s), individually or in combination, can update a buffer of acceleration samples. The computing device(s) can update the buffer via one or more components within the subject detection module 994, for example. Existing acceleration samples in the buffer (or cache or another type of storage device) constitute a buffered acceleration waveform. The buffered acceleration waveform may include pairs having a conditioned (or preprocessed) acceleration sample value and a timestamp or another type of datum (e.g., a natural number defining an index) indicative of a time corresponding to the acceleration sample value. Updating the buffer of acceleration samples includes adding a conditioned (or preprocessed) current chunk of acceleration samples to the buffered acceleration waveform. The current chunk of acceleration samples also may be referred to as the latest chunk of acceleration samples. Simply as an illustration, diagram 1010 in FIG. 10 depicts existing acceleration samples in buffer as a first block 1020a and the conditioned current chunk of acceleration sample as a second block 1020b. The updated buffer of conditioned acceleration samples corresponds to the union of block 102a and block 1020b.

At stage 930, the computing device(s), individually or in combination, can partition the updated buffered acceleration waveform into two or more intervals. The computing device(s) can update the buffered acceleration waveform via one or more components within the subject detection module 994, for example. The two or more intervals may be overlapping in some cases. The intervals may be defined by a first parameter and a second parameter. The first parameter may be referred to as “interval length” and the second parameter may be referred to as “interval stride.” The interval length defines a span of an interval, where the span is defined as number of immediately consecutive acceleration samples within the buffer. The interval stride defines a degree of overlap between consecutive intervals. The relative shift is defined as another number of immediately consecutive acceleration samples. The interval length may be greater than the interval stride. In some cases, each one of the two or more intervals has a same interval length. Simply as an illustration, diagram 1040 in FIG. 10 depicts an example of a partition of the updated buffer shown in diagram 1010 into a first interval 1050a and a second interval 1050b, each having a same interval length 1060. An interval stride 1070 defines a degree of overlap between the first interval 1050a and the second interval 1050b.

At stage 940, the computing device(s), individually or in combination, can determine waveform features for an interval resulting from the partition at stage 930. The computing device(s) can update the buffered acceleration waveform via one or more components within the subject detection module 994, for example. Examples of waveform features include spectral entropy, total amount of signal energy, and signal energy in bands corresponding to typical respiration rate and typical heart rate. The total amount of signal energy may be determined across all bands, e.g., respiration and cardiac function. Thus, such a waveform feature may be very large during movement and/or disturbances associated with a bed in accordance with aspects of this disclosure, and smaller in other situations. The total amount of signal energy may be an unbounded value.

Further, an example of a frequency band that may be used to determine signal energy for RR is about 0.0833 Hz to about 0.333 Hz, which band corresponds to about five to about 20 breaths per minute. An example of a frequency band that may be used to determine signal energy for HR is about 0.9167 Hz to about 1.670 Hz, which band corresponds to about 55 to about 100 beats per minute. Respective values of signal energy in the RR band and signal energy in the HR band may be divided by the total amount of signal energy across all bands. Such a normalization results in values of the signal energy in RR band and signal energy in the HR band to be between 0 and 1. As a result, those waveform features may be generalized across signals of different magnitudes.

Without intending to be bound by theory and/or modeling, spectral entropy provides a measure of regularity or randomness in the signal. When a subject is present in a bed in accordance with this disclosure, the signal is more regular, and is dominated by RR and HR components. As a result, the signal has lower entropy. When no subject is present in the bed, the signal is pure noise, and thus more random. Hence, the signal has higher entropy.

At stage 950, the computing device(s), individually or in combination, can determine a classification attribute (or classifier) for the interval for which waveform features have been determined at stage 940. The classification attribute characterizes the interval as corresponding to a subject in bed or to subject not in bed. In other words, the classification attribute assigns the interval a status indicative of “subject in bed” or “subject not in bed.” The computing device(s) can determine or otherwise generate the classification attribute by applying the predictive model 998 to the waveform features determined at stage 940, for example.

At stage 960, the computing device(s), individually or in combination, can determine if another interval is to be processed. The computing device(s) can perform such a determination via one or more components within the subject detection module 994, for example. An affirmative determination causes the computing device(s) to implement stage 940 and stage 950 for the next interval. A negative determination causes the flow to continue to stage 970, where the computing device(s), individually or in combination, determine a final in-bed status using the classifier stream obtained for the multiple intervals associated with an updated buffer of acceleration samples. The classifier stream is a time series of determined classification attributes. Each classification attribute in the time series corresponds to a respective interval of the multiple intervals, and has a value assigning the respective interval a status indicative of either “subject in bed” or “subject not in bed.” The computing device(s) can determine the final in-bed status via one or more components within the subject detection module 994, for example.

In some cases, computing device(s) that implement the stage 960 can determine the final in-bed status corresponding to the updated buffer of acceleration signals by removing classification attributes from the classifier stream. A classification attribute can be removed by applying one or more filtering rules directed to suppressing spurious predictions that have been made at stage 950. Because each classification attribute designates an interval as having a “subject in bed” status or a “subject not in bed” status, two filtering rules can be configured and used to remove classifier attributes. A first filtering rule can dictate that a classifier attribute designating “subject not in bed” status for an interval is switched to designate “subject in bed” status for the interval when the interval is less than a first defined period. A second filtering rule can dictate that a classifier designating “subject in bed” status for an interval is switched to designate “subject not in bed” status for the interval when the interval is less than a second defined time period.

In one example configuration, the first defined period is 15 seconds (15 s) and the second defined period is eight seconds (8 s). Such an example configuration causes transitions from “not in bed” to “in bed” status to be easier to occur than transitions from “in bed” to “not in bed” status. In this way, estimation of virtual sign(s) are less likely to be avoided while a subject is in a bed in accordance with aspects of this disclosure.

At stage 980, the computing device(s), individually or in combination, can cause one or more devices to perform an action based on the final in-bed status. The computing device(s) can cause, via one or more components within the subject detection module 994, the device(s) to perform the action. The action can be any of the actions described herein. In some cases, when a subject is determined to be absent from the bed, the action may include ceasing monitoring of vital signs in accordance with this disclosure. In those cases, the action also can include sending an electronic message to a computing device remotely located relative to the computing device(s) that perform the example process flow 900. Such a remote device may be a caregiver device or a clinician device, and the electronic message (and email or a text message) may inform a caregiver or clinician that the subject is not in the bed. In other cases, the action may include initiating or resuming monitoring of vital signs in accordance with this disclosure. In yet other cases, the action may include presenting an audible alarm, a visual alarm, and/or a haptic alarm to convey that a subject not in bed status has been determined. Many other actions can be implemented.

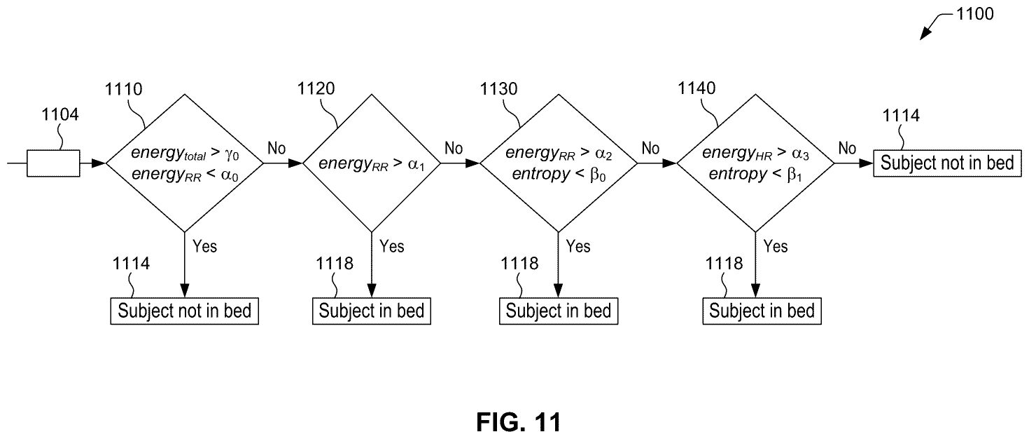

With respect to stage 950, there are numerous ways to configure a classification attribute that can designate a portion of acceleration magnitude samples as corresponding to “subject in bed” or “subject not in bed.” In one example, as is illustrated in FIG. 11, the computing device(s) that implement the stage 950 can operate on a portion 1104 of acceleration magnitude signal that has been conditioned as described in connection with stage 910 (FIG. 9A) to implement an example decision tree 1100 based on multiple threshold values for the waveform features including signal energy and spectral entropy. The example decision tree 1100 may be or may constitute the predictive model 998 (FIG. 9B). More specifically, as part of implementing the decision tree 1100, the computing device(s) can determine if total signal energy exceeds a first threshold value and signal energy in the RR band is less than a second threshold value, where the second threshold value is less than the first threshold value. In response to an affirmative determination (“Yes” branch), the decision tree 1100 designates the portion of acceleration signal magnitude as “subject not in bed.” Hence, the computing device(s) can configure a classification attribute as designating the portion 1104 as having a “subject not in bed” status 1114. In instances where such a condition is not satisfied (“No” branch), implementation of the decision tree 1100 continues to block 1120.

At block 1120, the computing device(s) can determine if the signal energy in the RR band in the portion 1104 exceeds a third threshold value. In response to an affirmative determination (“Yes” branch), the decision tree 1100 designates the portion 1104 of acceleration signal magnitude as “subject in bed.” Hence, the computing device(s) can configure a classification attribute to designate the portion 1104 as having a “subject in bed” status 1118. The “Yes” branch stemming from block 1120 indicates that there is strong signal in the RR band and, thus, a high probability of a subject being person in a bed in accordance with aspects of his disclosure. In instances where the signal energy in RR band does not exceed the third threshold value (“No” branch), further implementation of the decision tree 1100 continues to block 1130.

At block 1130, the computing device(s) can determine if, for the portion 1104, signal energy in the RR band exceeds a fourth threshold value (less than the third threshold value) and signal entropy is less than a fifth threshold value. In response to an affirmative determination (“Yes” branch), the decision tree 1100 designates the portion 1104 of acceleration signal magnitude as “subject in bed.” Hence, the computing device(s) can configure a classification attribute to designate the portion 1104 as having “subject in bed” status 1118. In instances where the signal energy in the RR band does not exceed the fourth threshold value and the signal entropy is equal to or greater than the first threshold value (“No” branch), further implementation of the decision tree 1100 continues to block 1140.

At block 1140, the computing device(s) can determine if, for the portion 1104, signal entropy is less than a sixth threshold value and signal energy in the HR band exceeds another threshold value. In response to an affirmative determination (“Yes” branch), the decision tree 1100 designates the portion 1104 of acceleration signal magnitude as “subject in bed.” Hence, the computing device(s) can configure a classification attribute to designate the portion 1104 as having “subject in bed” status 1118. In instances where the signal entropy is equal to or greater than the sixth threshold value and the signal energy in the HR band does not exceed the another threshold value (“No” branch), further implementation of the decision tree 1100 designates the portion 1104 of acceleration signal magnitude as “subject not in bed.” Hence, the computing device(s) can configure a classification attribute to designate the portion 1104 as having “subject not in bed” status 1114.

Some of the foregoing threshold values can have respective values that range from 0 to 1. At least one other threshold value can have respective values that are bound from below. The thresholds values can satisfy configurable appropriate relationships among themselves that permit implementing the example decision tree 1100 described above.

In addition or in some cases, a random forest (RF) classifier combined with an HR detection module can provide a classification attribute that designates a portion of a conditioned acceleration magnitude signal as “subject in bed” or “subject not in bed.” The random forest (RF) classifier individually may provide greater prediction accuracy than a decision tree, such as the decision tree 1100 described above. In order to reduce false positive “subject in bed” designations, the HR detection module can be combined with the random forest classifier, to assess if the portion of a conditioned acceleration magnitude signal includes signal contributions from HR. The HR detection module thus provides a safeguard against mispredictions of “subject not in bed.”

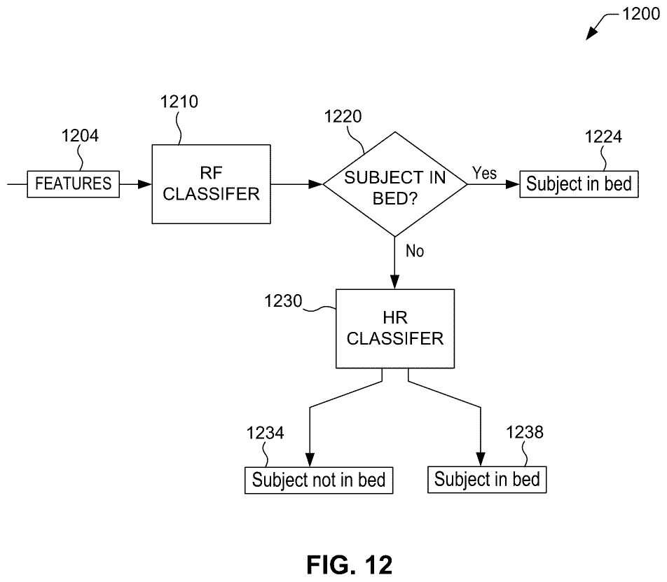

FIG. 12 is a schematic block diagram of an example predictive model 1200 based on a combination of an RF classifier 1210 and an HR classifier 1230, in accordance with one or more aspects of this disclosure. The example predictive model 1200 may be or may constitute the predictive model 998 (FIG. 9). The RF classifier 1210 can receive waveform features 1204. The waveform features 1204 may include, for example, a combination of spectral entropy, total amount of signal energy, signal energy in RR band, and signal energy in HR band. The RF classifier 120 can generate a prediction indicative of one of “subject in bed” or in subject not in bed.” The example predictive model 1200 can determine at block 1220 if the prediction from the RF classifier is indicative of “subject in bed.” In response to an affirmative determination, the example predictive model 1200 outputs a classification attribute 1224 indicative of “subject in bed.” In the alternative, in response to a negative determination at block 1220, the example predictive model 1200 can at least some of the waveform features 1204 to the HR classifier 1230. The HR classifier 1230 can then output one of a classifier attribute 1234 indicative of “subject not in bed” or another classifier indicative of “subject in bed.” The output of the HR classifier constitutes the output of the predictive model 1200.

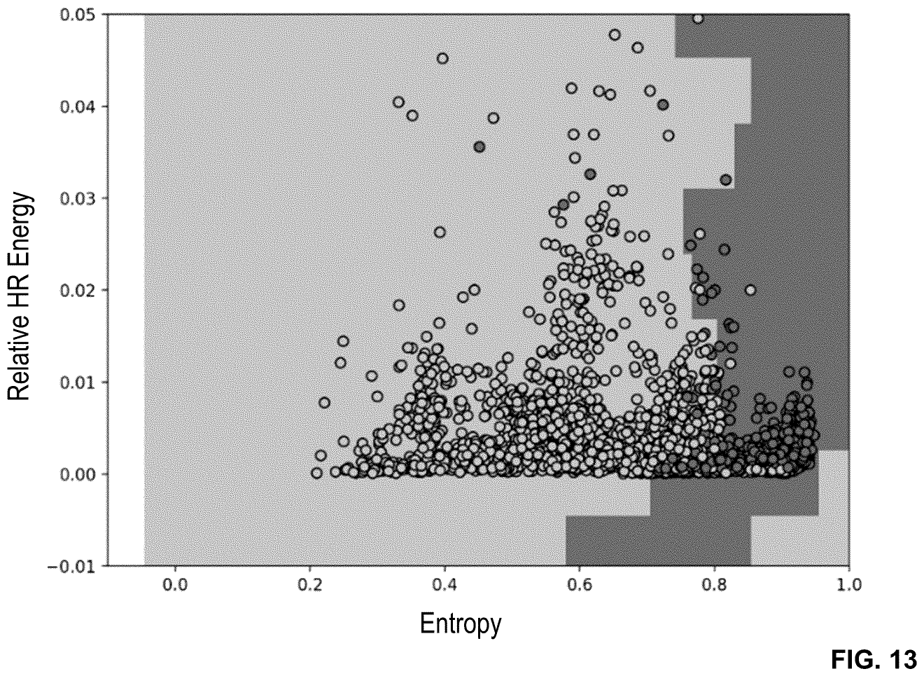

In some configurations, the HR detection module provides a HR classifier 1230 that can be implemented as a support vector machine (SVM) using two features: signal entropy and ratio of signal energy in HR band to total signal energy. In one example, the SVM is formed with an RBF kernel and degree 3. This disclosure, however, is not limited in that respect. FIG. 13 illustrates a prediction map for the HR classifier 1230 implemented using a SVM with signal entropy and ratio of signal energy in HR band to total signal energy as features. The prediction map illustrates decision boundaries between “subject in bed” status and “subject not in bed” status. Open circles indicate “subject in bed” status and solid circles indicate “subject not in bed” status. The HR classifier 1230 based on SVM may permit classifying strong HR pulses regardless of RR signal presence.

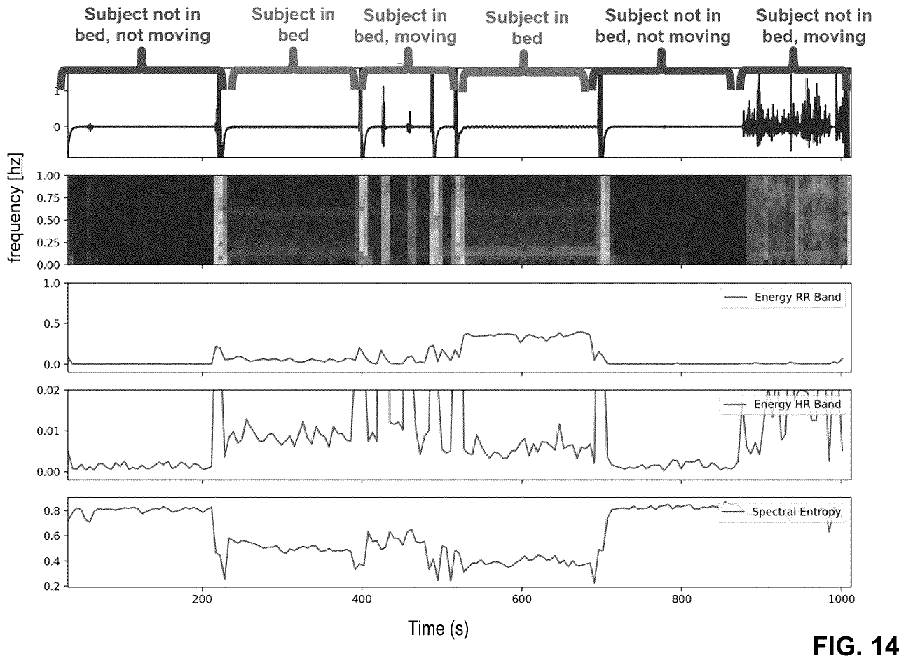

FIG. 14 illustrates how the waveform features can be useful for detection of a subject in accordance with aspects of this disclosure. Spectral entropy dips when a subject (e.g., a person) is in bed, as the signal becomes less random. Relative RR and HR energies increase when a subject is in bed. As described herein, entropy and relative energy in particular bands may be useful features because they range from [0,1] which may aid with generalization across subjects and environments.

Various example embodiments that emerge from the description herein and the annexed figures include the following Examples.

Example 1. A computer-implemented method, comprising: conditioning acceleration signals corresponding to respective accelerometer channels of an accelerometer device mechanically coupled with a subject via a solid medium, resulting in conditioned acceleration signals; generating, using the conditioned acceleration signals, a conditioned acceleration magnitude signal representing of one of pulmonary ventilation or blood circulation; determining, using the conditioned acceleration magnitude signal, one or more of a first dataset indicative of peaks in the conditioned acceleration magnitude signal, or a second dataset indicative of troughs in the conditioned acceleration magnitude signal; determining, using one or both of the first dataset or the second dataset, multiple estimates of a vital sign of the subject; determining, using the multiple estimates, an observed estimate of the vital sign; and providing the observed estimate of the vital sign.

Example 2. The computer-implemented method of Example 1, wherein the providing comprises causing a display device to present the observed estimate of the vital sign at a defined presentation rate.

Example 3. The computer-implemented method of Example 1, wherein the conditioning comprises detrending a first accelerometer signal of the accelerometer signals, resulting in a first detrended accelerometer signal.

Example 4. The computer-implemented method of Example 3, wherein the conditioning further comprises filtering the first detrended accelerometer signal.

Example 5. The computer-implemented method of Example 4, wherein the vital sign is respiration rate, and wherein the filtering comprises applying a low-pass filter having a cutoff frequency of about 0.2 Hz.

Example 6. The computer-implemented method of Example 4, wherein the vital sign is heart rate, and wherein the filtering comprises applying a band-pass filter having a lower cutoff frequency of about 1.0 Hz and a higher cutoff frequency of about 20 Hz.

Example 7. The computer-implemented method of Example 1, wherein the determining, using one or both of the first dataset or the second dataset, the multiple estimates of the vital sign of the subject comprises, determining, using the peak dataset and the trough dataset, time intervals corresponding to respective consecutive peak-trough-peak triples, with each one of the time intervals providing a respective rate associated with the vital sign; determining rates corresponding to respective inverses of the time intervals; and configuring the rates as respective ones of the multiple estimates.

Example 8. The computer-implemented method of Example 1, wherein the determining, using the multiple estimates, the observed estimate of the vital sign comprises, combining at least a subset of the multiple estimates by determining an average of at least the subset of the multiple estimates over a defined moving time interval; and configuring the average as the observed estimate of the vital sign.

Example 9. A computing device, comprising: at least one processor; and at least one memory device storing processor-executable instructions that, in response to execution by the at least one processor, individually or in combination, cause the device at least to, condition acceleration signals corresponding to respective accelerometer channels of an accelerometer device mechanically coupled with a subject via a solid medium, resulting in conditioned acceleration signals; generate, using the conditioned acceleration signals, a conditioned acceleration magnitude signal representing of one of pulmonary ventilation or blood circulation; determine, using the conditioned acceleration magnitude signal, one or more of a first dataset indicative of peaks in the conditioned acceleration magnitude signal, or a second dataset indicative of troughs in the conditioned acceleration magnitude signal; determine, using one or both of the first dataset or the second dataset, multiple estimates of a vital sign of the subject; determine, using the multiple estimates, an observed estimate of the vital sign; and provide the observed estimate of the vital sign.

Example 10. The computing device of Example 9, wherein providing the observed estimate of the vital sign comprises causing a display device to present the observed estimate of the vital sign at a defined presentation rate.

Example 11. The computing device of Example 9, wherein conditioning the acceleration signals comprises detrending a first accelerometer signal of the accelerometer signals, resulting in a first detrended accelerometer signal.

Example 12. The computing device of Example 11, wherein conditioning the acceleration signals further comprises filtering the first detrended accelerometer signal.

Example 13. The computing device of Example 9, wherein determining, using one or both of the first dataset or the second dataset, the multiple estimates of the vital sign of the subject comprises, determining, using the peak dataset and the trough dataset, time intervals corresponding to respective consecutive peak-trough-peak triples, with each one of the time intervals providing a respective rate associated with the vital sign; determining rates corresponding to respective inverses of the time intervals; and configuring the rates as respective ones of the multiple estimates.

Example 14. The computing device of Example 9, wherein determining, using the multiple estimates, the observed estimate of the vital sign comprises, combining at least a subset of the multiple estimates by determining an average of at least the subset of the multiple estimates over a defined moving time interval; and configuring the average as the observed estimate of the vital sign.

Example 15. A system, comprising: an accelerometer device configured to generate acceleration signals in respective measurement channels, with the accelerometer device being mechanically coupled with a subject via a solid medium; and a computing device comprising, at least one processor; and at least one memory device storing processor-executable instructions that, in response to execution by the at least one processor, individually or in combination, cause the computing device at least to, condition the acceleration signals in the respective accelerometer channels, resulting in conditioned acceleration signals; generate, using the conditioned acceleration signals, a conditioned acceleration magnitude signal representing of one of pulmonary ventilation or blood circulation; determine, using the conditioned acceleration magnitude signal, one or more of a first dataset indicative of peaks in the conditioned acceleration magnitude signal, or a second dataset indicative of troughs in the conditioned acceleration magnitude signal; determine, using one or both of the first dataset or the second dataset, multiple estimates of a vital sign of the subject; determine, using the multiple estimates, an observed estimate of the vital sign; and provide the observed estimate of the vital sign.

Example 16. The system of Example 15, wherein providing the observed estimate of the vital sign comprises causing a display device to present the observed estimate of the vital sign at a defined presentation rate.

Example 17. The system of Example 15, wherein conditioning the acceleration signals comprises detrending a first accelerometer signal of the accelerometer signals, resulting in a first detrended accelerometer signal.

Example 18. The system of Example 17, wherein conditioning the acceleration signals further comprises filtering the first detrended accelerometer signal.

Example 19. The system of Example 15, wherein determining, using one or both of the first dataset or the second dataset, the multiple estimates of the vital sign of the subject comprises, determining, using the peak dataset and the trough dataset, time intervals corresponding to respective consecutive peak-trough-peak triples, with each one of the time intervals providing a respective rate associated with the vital sign; determining rates corresponding to respective inverses of the time intervals; and configuring the rates as respective ones of the multiple estimates.

Example 20. The system of Example 15, wherein determining, using the multiple estimates, the observed estimate of the vital sign comprises, combining at least a subset of the multiple estimates by determining an average of at least the subset of the multiple estimates over a defined moving time interval; and configuring the average as the observed estimate of the vital sign.

Example 21. A computer-implemented method, comprising: conditioning a current group of acceleration magnitude samples, resulting in a current group of conditioned acceleration magnitude samples; updating a buffer to add the current group of conditioned acceleration magnitude samples, with the buffer containing a prior group of conditioned acceleration magnitude samples, resulting in a cumulative group of conditioned acceleration magnitude samples; partitioning the cumulative group of conditioned acceleration magnitude sample into multiple intervals of conditioned acceleration magnitude samples; generating a stream of multiple classification attributes by applying a predictive model to the multiple intervals of conditioned acceleration magnitude samples, with each classification attribute of the multiple classification attributes designating a respective interval as having one of a first status indicative of presence of a subject in a bed or a second status indicative of absence of the subject in the bed; determining, using the stream of multiple classification attributes, an in-bed status indicative of one presence of the subject in the bed or absence of the subject in the bed; and causing one or more devices to perform an action based on the in-bed status.

Example 22. The computer-implemented method of Example 21, wherein conditioning the current group of acceleration magnitude samples comprises detrending the current group of acceleration magnitude samples by applying an infinite-impulse response high-pass filter to the current group.

Example 23. The computer-implemented method of Example 21, further comprising generating one or more waveform features for a first interval of the multiple intervals of conditioned acceleration magnitude samples.

Example 24. The computer-implemented method of Example 23, wherein each of the one or more features comprises one of a signal spectral entropy, total amount of signal energy, signal energy in a frequency band corresponding to a respiration rate, signal energy in a frequency band corresponding to heart rate.

Example 25. The computer-implemented method of Example 23, wherein the applying the predictive model to the multiple intervals of conditioned acceleration magnitude samples comprises applying the predictive model to the one or more waveform features for the first interval, resulting in a first classification attribute.

Example 26. The computer-implemented method of Example 21, wherein the predictive model comprises a decision tree.

Example 27. The computer-implemented method of Example 21, wherein the predictive model comprises a first classifier model and a second classifier model, the first classifier model configured to operate on one or a combination of waveform features including spectral entropy, total amount of signal energy, or signal energy in a frequency band corresponding to a respiration rate; and the second classifier model configured to operate at least one waveform feature including signal energy in a frequency band corresponding to heart rate.

Example 28. The computer-implemented method of Example 27, wherein the first classifier model comprises a random forest classifier, and wherein the second classifier model comprises a support vector machine (SVM) model.

Example 29. The computer-implemented method of Example 28, wherein the SVM model is configured to operate on the spectral entropy and the signal energy in the frequency band corresponding to heart rate.

Example 30. A computing device, comprising: at least one processor; and at least one

memory device storing processor-executable instructions that, in response to execution by the at least one processor, individually or in combination, cause the computing device at least to, condition a current group of acceleration magnitude samples, resulting in a current group of conditioned acceleration magnitude samples; update a buffer to add the current group of conditioned acceleration magnitude samples, with the buffer containing a prior group of conditioned acceleration magnitude samples, resulting in a cumulative group of conditioned acceleration magnitude samples; partition the cumulative group of conditioned acceleration magnitude sample into multiple intervals of conditioned acceleration magnitude samples; generate a stream of multiple classification attributes by applying a predictive model to the multiple intervals of conditioned acceleration magnitude samples, with each classification attribute of the multiple classification attributes designating a respective interval as having one of a first status indicative of presence of a subject in a bed or a second status indicative of absence of the subject in the bed; determine, using the stream of multiple classification attributes, an in-bed status indicative of one presence of the subject in the bed or absence of the subject in the bed; and cause one or more devices to perform an action based on the in-bed status.

Example 31. The computing device of Example 30, wherein conditioning the current group of acceleration magnitude samples comprises detrending the current group of acceleration magnitude samples by applying an infinite-impulse response high-pass filter to the current group.

Example 32. The computing device of Example 30, with the at least one memory device storing further processor-executable instructions that, in response to execution by the at least one processor, individually or in combination, further cause the computing device at least to generate one or more waveform features for a first interval of the multiple intervals of conditioned acceleration magnitude samples.

Example 33. The computing device of Example 32, wherein each of the one or more features comprises one of a signal spectral entropy, total amount of signal energy, signal energy in a frequency band corresponding to a respiration rate, signal energy in a frequency band corresponding to heart rate.

Example 34. The computing device of Example 32, wherein applying the predictive model to the multiple intervals of conditioned acceleration magnitude samples comprises applying the predictive model to the one or more waveform features for the first interval, resulting in a first classification attribute.

Example 35. The computing device of Example 30, wherein the predictive model comprises a decision tree.

Example 36. The computing device of Example 30, wherein the predictive model comprises a first classifier model and a second classifier model, the first classifier model configured to operate on one or a combination of waveform features including spectral entropy, total amount of signal energy, or signal energy in a frequency band corresponding to a respiration rate; and the second classifier model configured to operate at least one waveform feature including signal energy in a frequency band corresponding to heart rate.

Example 37. A system, comprising: an accelerometer device configured to generate acceleration signals corresponding to respective measurement channels; and a computing device comprising, at least one processor; and at least one memory device storing processor-executable instructions that, in response to execution by the at least one processor, individually or in combination, cause the computing device at least to, condition a current group of acceleration magnitude samples, resulting in a current group of conditioned acceleration magnitude samples; update a buffer to add the current group of conditioned acceleration magnitude samples, with the buffer containing a prior group of conditioned acceleration magnitude samples, resulting in a cumulative group of conditioned acceleration magnitude samples; partition the cumulative group of conditioned acceleration magnitude sample into multiple intervals of conditioned acceleration magnitude samples; generate a stream of multiple classification attributes by applying a predictive model to the multiple intervals of conditioned acceleration magnitude samples, with each classification attribute of the multiple classification attributes designating a respective interval as having one of a first status indicative of presence of a subject in a bed or a second status indicative of absence of the subject in the bed; determine, using the stream of multiple classification attributes, an in-bed status indicative of one presence of the subject in the bed or absence of the subject in the bed; and cause one or more devices to perform an action based on the in-bed status.

Example 38. The system of Example 37, with the at least one memory device storing further processor-executable instructions that, in response to execution by the at least one processor, individually or in combination, further cause the computing device at least to generate one or more waveform features for a first interval of the multiple intervals of conditioned acceleration magnitude samples.

Example 39. The system of Example 38, wherein each of the one or more features comprises one of a signal spectral entropy, total amount of signal energy, signal energy in a frequency band corresponding to a respiration rate, signal energy in a frequency band corresponding to heart rate.

Example 40. The system of Example 39, wherein applying the predictive model to the multiple intervals of conditioned acceleration magnitude samples comprises applying the predictive model to the one or more waveform features for the first interval, resulting in a first classification attribute.

Example 41. A system, comprising: an accelerometer device configured to generate acceleration signals corresponding to respective measurement channels, with the accelerometer device mechanically coupled with a subject via a solid medium; and at least one processor; and at least one memory device storing processor-executable instructions that, in response to execution by the at least one processor, individually or in combination, cause the at least one processor at least to, condition the acceleration signals, resulting in conditioned acceleration signals; generate, using the conditioned acceleration signals, a conditioned acceleration magnitude signal representing of one of pulmonary ventilation or blood circulation; determine, using the conditioned acceleration magnitude signal, one or more of a first dataset indicative of peaks in the conditioned acceleration magnitude signal, or a second dataset indicative of troughs in the conditioned acceleration magnitude signal; determine, using one or both of the first dataset or the second dataset, multiple estimates of a vital sign of the subject; determine, using the multiple estimates, an observed estimate of the vital sign; and cause a display device to present the observed estimate of the vital sign.

Example 42. The system of Example 41, wherein conditioning the acceleration signals comprises detrending a first accelerometer signal of the accelerometer signals, resulting in a first detrended accelerometer signal.

Example 43. The system of Example 42, wherein conditioning the acceleration signals further comprises filtering the first detrended accelerometer signal.

Example 44. The system of Example 41, wherein determining, using one or both of the first dataset or the second dataset, the multiple estimates of the vital sign of the subject comprises, determining, using the peak dataset and the trough dataset, time intervals corresponding to respective consecutive peak-trough-peak triples, with each one of the time intervals providing a respective rate associated with the vital sign; determining rates corresponding to respective inverses of the time intervals; and configuring the rates as respective ones of the multiple estimates.

Example 45. The system of Example 41, wherein determining, using the multiple estimates, the observed estimate of the vital sign comprises, combining at least a subset of the multiple estimates by determining an average of at least the subset of the multiple estimates over a defined moving time interval; and configuring the average as the observed estimate of the vital sign.

Example 46. The system of Example 41, wherein the display device is integrated into one of a user device associated with the subject or a clinician device that is part of a system of healthcare facilities.

Example 47. The system of Example 41, with the accelerometer device and the at least one processor being integrated into a wearable device configured to be mounted on the torso of the subject.

Example 48. The system of Example 41, with the accelerometer device being integrated into a wearable device configured to be mounted on the torso of the subject, and with the at least one processor being integrated into a computing device communicatively coupled with the wearable device.

Example 49. The system of Example 41, wherein the display device is integrated into a user device, and wherein causing the display device to present the observed estimate comprises sending, by the computing device, via at least one or more communication networks, data defining the observed estimate of the vital sign to the user device.

Example 50. The system of Example 41, with the accelerometer device being integrated into a wearable device configured to be mounted on the torso of the subject, and with the at least one processor being integrated into one or more processing platform devices remotely located relative to the wearable device.

Example 51. A wearable device, comprising: an accelerometer device configured to generate acceleration signals corresponding to respective measurement channels, with the accelerometer device mechanically coupled with a subject via a solid medium; at least one processor; and at least one memory device storing processor-executable instructions that, in response to execution by the at least one processor, individually or in combination, cause the wearable device at least to; condition the acceleration signals, resulting in conditioned acceleration signals; generate, using the conditioned acceleration signals, a conditioned acceleration magnitude signal representing of one of pulmonary ventilation or blood circulation; determine, using the conditioned acceleration magnitude signal, one or more of a first dataset indicative of peaks in the conditioned acceleration magnitude signal, or a second dataset indicative of troughs in the conditioned acceleration magnitude signal; determine, using one or both of the first dataset or the second dataset, multiple estimates of a vital sign of the subject; determine, using the multiple estimates, an observed estimate of the vital sign; and cause a display device to present the observed estimate of the vital sign.

Example 52. The wearable device of Example 50, wherein determining, using one or both of the first dataset or the second dataset, the multiple estimates of the vital sign of the subject comprises, determining, using the peak dataset and the trough dataset, time intervals corresponding to respective consecutive peak-trough-peak triples, with each one of the time intervals providing a respective rate associated with the vital sign; determining rates corresponding to respective inverses of the time intervals; and configuring the rates as respective ones of the multiple estimates.

Example 53. The wearable device of Example 51, wherein determining, using the multiple estimates, the observed estimate of the vital sign comprises, combining at least a subset of the multiple estimates by determining an average of at least the subset of the multiple estimates over a defined moving time interval; and configuring the average as the observed estimate of the vital sign.

Example 54. The wearable device of Example 51 being configured to be mounted on the torso of a subject.

Example 55. The wearable device of Example 51, wherein the display device is integrated into one of a user device associated with the subject, and wherein causing the display device to present the observed estimate comprises sending, by the wearable device, via a wireless communication link, data defining the observed estimate of the vital sign to the user device.

Example 56. The wearable device of Example 51, further comprising one or more lighting devices to convey the observed estimate of the vital sign.

Example 57. The wearable device of Example 56, with the one or more lighting devices comprising, a first lighting device to configured to emit light of a first color to communicate that the observed estimate of the vital sign is outside a safe zone; a second lighting device configured to emit light of a second color to communicate that the observed estimate of the vital sign is within a defined range of an expected range of values; and a third lighting device configured to emit light of a third color to communicate that the observed estimate of the vital sign is in an unsafe range.

Various aspects of the disclosure may take the form of an entirely or partially hardware aspect, an entirely or partially software aspect, or a combination of software and hardware. Furthermore, as described herein, various aspects of the disclosure (e.g., systems and methods) may take the form of a computer program product comprising a computer-readable non-transitory storage medium having processor-accessible instructions (e.g., computer-readable and/or computer-executable instructions) such as computer software, encoded or otherwise embodied in such storage medium. Those instructions can be read or otherwise accessed and executed by one or more processors, individually or in combination, to perform or permit the performance of the operations described herein. The instructions can be provided in any suitable form, such as source code, compiled code, interpreted code, executable code, static code, dynamic code, assembler code, combinations of the foregoing, and the like. Any suitable computer-readable non-transitory storage medium may be utilized to form the computer program product. For instance, the computer-readable medium may include any tangible non-transitory medium for storing information in a form readable or otherwise accessible by one or more computers or processor(s) functionally coupled thereto. Non-transitory storage media can include read-only memory (ROM); random access memory (RAM); magnetic disk storage media; optical storage media; flash memory, and so forth.

Aspects of this disclosure are described herein with reference to block diagrams and flowchart illustrations of processor-implemented methods, systems, devices, apparatuses, and computer program products. It can be understood that each block of the block diagrams and flowchart illustrations, and combinations of blocks in the block diagrams and flowchart illustrations, respectively, can be implemented by processor-accessible instructions. Such instructions may include, for example, computer program instructions (e.g., processor-readable and/or processor-executable instructions). The processor-accessible instructions may be built (e.g., linked and compiled) and retained in processor-executable form in one or multiple memory devices or one or many other processor-accessible non-transitory storage media. These computer program instructions also can be stored in a processor-readable memory, where in response to execution by one or more processors, individually or in combination, the computer program instructions can direct a computer, a computing device, or other programmable data processing apparatus to function in a particular manner, such that the instructions stored in the processor-readable memory produce an article of manufacture including processor-accessible instructions (e.g., processor-readable instructions and/or processor-executable instructions) to implement the function specified in the flowchart blocks (individually or in a particular combination) or blocks in block diagrams (individually or in a particular combination). The computer program instructions can be loaded onto a computer, a computing device, or other programmable data processing apparatus to cause a series of operations to be performed on the computer or other programmable apparatus to produce a computer-implemented process. The series of operations may be performed in response to execution by one or more processor or other types of processing circuitry. Thus, such instructions that execute on the computer or other programmable apparatus provide operations for implementing the functions specified in the flowchart blocks (individually or in a particular combination) or blocks in block diagrams (individually or in a particular combination).

In some implementations, the processor-accessible instructions may be loaded or otherwise incorporated into a general purpose computer, a special purpose computer, or another programmable information processing apparatus to produce a particular machine, such that the operations or functions specified in the flowchart block or blocks can be implemented in response to execution at the computer or processing apparatus. More specifically, the loaded processor-accessible instructions may be accessed and executed by one or multiple processors, individually or in combination, or other types of processing circuitry. In response to execution, the loaded processor-accessible instructions provide the functionality described in connection with flowchart blocks (individually or in a particular combination) or blocks in block diagrams (individually or in a particular combination). Thus, such instructions which execute on a computer, a computing device, or other programmable data processing apparatus can create a means for implementing the functions specified in the flowchart blocks (individually or in a particular combination) or blocks in block diagrams (individually or in a particular combination).

Unless otherwise expressly stated, it is in no way intended that any protocol, procedure, process, or method set forth herein be construed as requiring that its acts or steps be performed in a specific order. Accordingly, where a process or method claim does not actually recite an order to be followed by its acts or steps or it is not otherwise specifically recited in the claims or descriptions of the subject disclosure that the steps are to be limited to a specific order, it is in no way intended that an order be inferred, in any respect. This holds for any possible non-express basis for interpretation, including: matters of logic with respect to the arrangement of steps or operational flow; plain meaning derived from grammatical organization or punctuation; the number or type of aspects described in the specification or annexed drawings; or the like.

As used in this disclosure, including the annexed drawings, the terms “component,” “module,” “interface,” “system,” and the like are intended to refer to a computer-related entity or an entity related to an apparatus with one or more specific functionalities. The entity can be either hardware, a combination of hardware and software, software, or software in execution. One or more of such entities are also referred to as “functional elements.” As an example, a component can be a process running on a processor, a processor, an object, an executable, a thread of execution, a program, and/or a computer. For example, both an application running on a server or network controller, and the server or network controller can be a component. One or more components can reside within a process and/or thread of execution and a component can be localized on one computer and/or distributed between two or more computers. Also, these components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which parts can be controlled or otherwise operated by program code executed by a processor. As yet another example, a component can be an apparatus that provides specific functionality through electronic components without mechanical parts, the electronic components can include a processor to execute program code that provides, at least partially, the functionality of the electronic components. As still another example, interface(s) can include I/O components or Application Programming Interface (API) components. While the foregoing examples are directed to aspects of a component, the exemplified aspects or features also apply to a system, module, and similar.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in this specification and annexed drawings should be construed to mean “one or more” unless specified otherwise or clear from context to be directed to a singular form.

In addition, the terms “example” and “such as” are utilized herein to mean serving as an instance or illustration. Any aspect or design described herein as an “example” or referred to in connection with a “such as” clause is not necessarily to be construed as preferred or advantageous over other aspects or designs described herein. Rather, use of the terms “example” or “such as” is intended to present concepts in a concrete fashion. The terms “first,” “second,” “third,” and so forth, as used in the claims and description, unless otherwise clear by context, is for clarity only and doesn't necessarily indicate or imply any order in time or space.

The term “processor,” as utilized in this disclosure, can refer to any computing processing unit or device comprising processing circuitry that can operate on data and/or signaling. A computing processing unit or device can include, for example, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can include an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. In some cases, processors can exploit nano-scale architectures, such as molecular and quantum-dot based transistors, switches and gates, in order to optimize space usage or enhance performance of user equipment. A processor may also be implemented as a combination of computing processing units.

In addition, terms such as “store,” “data store,” data storage,” “database,” and substantially any other information storage component relevant to operation and functionality of a component, refer to “memory components,” or entities embodied in a “memory” or components comprising the memory. It will be appreciated that the memory components described herein can be either volatile memory or nonvolatile memory, or can include both volatile and nonvolatile memory. Moreover, a memory component can be removable or affixed to a functional element (e.g., device, server).

Simply as an illustration, nonvolatile memory can include read only memory (ROM), programmable ROM (PROM), electrically programmable ROM (EPROM), electrically erasable ROM (EEPROM), or flash memory. Volatile memory can include random access memory (RAM), which acts as external cache memory. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). Additionally, the disclosed memory components of systems or methods herein are intended to comprise, without being limited to comprising, these and any other suitable types of memory.

Various aspects described herein can be implemented as a method, system, device, apparatus, or article of manufacture using standard programming and/or engineering techniques. In addition, various of the aspects disclosed herein also can be implemented by means of program modules or other types of computer program instructions stored in a memory device and executed by a processor, or other combination of hardware and software, or hardware and firmware. Such program modules or computer program instructions can be loaded onto a general purpose computer, a special purpose computer, or another type of programmable data processing apparatus to produce a machine, such that the instructions which execute on the computer or other programmable data processing apparatus create a means for implementing the functionality of disclosed herein.

The term “article of manufacture” as used herein is intended to encompass a computer program accessible from any computer-readable device, carrier, or media. For example, computer readable media can include but are not limited to magnetic storage devices (e.g., hard drive disk, floppy disk, magnetic strips, or similar), optical discs (e.g., compact disc (CD), digital versatile disc (DVD), blu-ray disc (BD), or similar), smart cards, and flash memory devices (e.g., card, stick, key drive, or similar).

What has been described above includes examples of one or more aspects of the disclosure. It is, of course, not possible to describe every conceivable combination of components or methodologies for purposes of describing these examples, and it can be recognized that many further combinations and permutations of the present aspects are possible. Accordingly, the aspects disclosed and/or claimed herein are intended to embrace all such alterations, modifications and variations that fall within the spirit and scope of the detailed description and the appended claims. Furthermore, to the extent that one or more of the terms “includes,” “including,” “has,” “have,” or “having” is used in either the detailed description or the claims, such term is intended to be inclusive in a manner similar to the term “comprising” as “comprising” is interpreted when employed as a transitional word in a claim.

What is claimed is:

1. A system, comprising:
an accelerometer device configured to generate acceleration signals corresponding to respective measurement channels, with the accelerometer device mechanically coupled with a subject via a solid medium; and
at least one processor; and
at least one memory device storing processor-executable instructions that, in response to execution by the at least one processor, individually or in combination, cause the at least one processor at least to, condition the acceleration signals, resulting in conditioned acceleration signals;
generate, using the conditioned acceleration signals, a conditioned acceleration magnitude signal representing of one of pulmonary ventilation or blood circulation;
determine, using the conditioned acceleration magnitude signal, one or more of a first dataset indicative of peaks in the conditioned acceleration magnitude signal, or a second dataset indicative of troughs in the conditioned acceleration magnitude signal;
determine, using one or both of the first dataset or the second dataset, multiple estimates of a vital sign of the subject;
determine, using the multiple estimates, an observed estimate of the vital sign; and
cause a display device to present the observed estimate of the vital sign.

2. The system of claim 1, wherein conditioning the acceleration signals comprises detrending a first accelerometer signal of the accelerometer signals, resulting in a first detrended accelerometer signal.

3. The system of claim 2, wherein conditioning the acceleration signals further comprises filtering the first detrended accelerometer signal.

4. The system of claim 1, wherein determining, using one or both of the first dataset or the second dataset, the multiple estimates of the vital sign of the subject comprises,
determining, using the peak dataset and the trough dataset, time intervals corresponding to respective consecutive peak-trough-peak triples, with each one of the time intervals providing a respective rate associated with the vital sign;
determining rates corresponding to respective inverses of the time intervals; and
configuring the rates as respective ones of the multiple estimates.

5. The system of claim 1, wherein determining, using the multiple estimates, the observed estimate of the vital sign comprises,
combining at least a subset of the multiple estimates by determining an average of at least the subset of the multiple estimates over a defined moving time interval; and
configuring the average as the observed estimate of the vital sign.

6. The system of claim 1, wherein the display device is integrated into one of a user device associated with the subject or a clinician device that is part of a system of healthcare facilities.

7. The system of claim 1, with the accelerometer device and the at least one processor being integrated into a wearable device configured to be mounted on the torso of the subject.

8. The system of claim 1, with the accelerometer device being integrated into a wearable device configured to be mounted on the torso of the subject, and
with the at least one processor being integrated into a computing device communicatively coupled with the wearable device.

9. The system of claim 8, wherein the display device is integrated into a user device, and wherein causing the display device to present the observed estimate comprises sending, by the computing device, via at least one or more communication networks, data defining the observed estimate of the vital sign to the user device.

10. The system of claim 1, with the accelerometer device being integrated into a wearable device configured to be mounted on the torso of the subject, and
with the at least one processor being integrated into one or more processing platform devices remotely located relative to the wearable device.

11. A wearable device, comprising:
an accelerometer device configured to generate acceleration signals corresponding to respective measurement channels, with the accelerometer device mechanically coupled with a subject via a solid medium;
at least one processor; and
at least one memory device storing processor-executable instructions that, in response to execution by the at least one processor, individually or in combination, cause the wearable device at least to;
condition the acceleration signals, resulting in conditioned acceleration signals;
generate, using the conditioned acceleration signals, a conditioned acceleration magnitude signal representing of one of pulmonary ventilation or blood circulation;
determine, using the conditioned acceleration magnitude signal, one or more of a first dataset indicative of peaks in the conditioned acceleration magnitude signal, or a second dataset indicative of troughs in the conditioned acceleration magnitude signal;
determine, using one or both of the first dataset or the second dataset, multiple estimates of a vital sign of the subject;
determine, using the multiple estimates, an observed estimate of the vital sign; and
cause a display device to present the observed estimate of the vital sign.

12. The wearable device of claim 11, wherein determining, using one or both of the first dataset or the second dataset, the multiple estimates of the vital sign of the subject comprises,
determining, using the peak dataset and the trough dataset, time intervals corresponding to respective consecutive peak-trough-peak triples, with each one of the time intervals providing a respective rate associated with the vital sign;
determining rates corresponding to respective inverses of the time intervals; and
configuring the rates as respective ones of the multiple estimates.

13. The wearable device of claim 11, wherein determining, using the multiple estimates, the observed estimate of the vital sign comprises,
combining at least a subset of the multiple estimates by determining an average of at least the subset of the multiple estimates over a defined moving time interval; and
configuring the average as the observed estimate of the vital sign.

14. The wearable device of claim 11 being configured to be mounted on the torso of a subject.

15. The wearable device of claim 11, wherein the display device is integrated into one of a user device associated with the subject, and wherein causing the display device to present the observed estimate comprises sending, by the wearable device, via a wireless communication link, data defining the observed estimate of the vital sign to the user device.

16. The wearable device of claim 11, further comprising one or more lighting devices to convey the observed estimate of the vital sign.

17. The wearable device of claim 16, with the one or more lighting devices comprising,
a first lighting device to configured to emit light of a first color to communicate that the observed estimate of the vital sign is outside a safe zone;
a second lighting device configured to emit light of a second color to communicate that the observed estimate of the vital sign is within a defined range of an expected range of values; and
a third lighting device configured to emit light of a third color to communicate that the observed estimate of the vital sign is in an unsafe range.
